(19) United States
(12) Patent Application Publication
(10) Pub. No.: US 2026/0137442 A1
(43) Pub. Date: May 21, 2026

(54) INTRAVASCULAR DENERVATION

(71) Applicant: Medtronic Ireland Manufacturing Unlimited Company, Dublin (IE)

(72) Inventors: Daniel Thomas Kollmann, Andover, MN (US); Matthew John Otto, Mankato, MN (US); Brian Allen Blow, Maple Grove, MN (US); Scott Raymond Smith, Chaska, MN (US)

(21) Appl. No.: 19/114,557

(22) PCT Filed: Sep. 29, 2023
(86) PCT No.: PCT/EP2023/077043

§ 371 Date: Mar. 24, 2025

Related U.S. Application Data
(60) Provisional application No. 63/377,908, filed on Sep. 30, 2022.

(51) Int. Cl. A61B 18/14; A61B 18/00; A61B 18/12

(52) U.S. Cl. CPC A61B 18/1492; A61B 18/1206; A61B 2018/0016; A61B 2018/0022; A61B 2018/00434; A61B 2018/00577; A61B 2018/00642; A61B 2018/00666; A61B 2018/00702; A61B 2018/00726; A61B 2018/00779; A61B 2018/00791; A61B 2018/00827; A61B 2018/00875; A61B 2018/00892; A61B 2018/1467

(57) ABSTRACT

Provided herein are expandable (e.g., inflatable by fluid) structures incorporating multiple electrodes for optimal circumferential denervation. In some examples, catheters incorporating such expandable structures may be used to ablate nerves in a perivascular space surrounding a vessel (e.g., to effect denervation of at least a portion of the liver, kidneys, pancreas and/or duodenum).

This application claims the benefit of and priority of U.S. Provisional Patent Application No. 63/377,908 , filed Sep. 30, 2022, and entitled “INTRAVASCULAR DENERVATION SYSTEMS, DEVICES AND METHODS,” the entire content of which is incorporated herein by reference.

### FIELD

This application is generally directed to systems, devices, and methods for tissue modulation (e.g., neuromodulation) and more particularly directed to systems, devices and methods for intravascular denervation.

### RELATED APPLICATIONS

The systems, devices, and methods described herein can be performed in conjunction with any of the methods or using any of the systems or devices described in U.S. Publication No. 2017/0348049 by Vrba et al. or WIPO Publication No. WO 2016/090175 naming inventors Vrba et al., each of which is hereby incorporated by reference herein in its entirety.

### BACKGROUND

Cardiovascular and metabolic regulation is controlled by both hormonal and neuronal signaling from the sympathetic nervous system in relevant organs such as kidney, liver, pancreas, skeletal muscle, and adipose tissue. Sympathetic nervous system overactivity is a key contributor to obesity, diabetes, hypertension, and other conditions. Excessive sympathetic nervous system activation results in chronic dysregulation of cardiometabolic processes and thereby contributes to the development of abnormalities and increased cardiovascular risk factors. The prevalence and degree of such disorders have markedly escalated over the recent decades. Ultimately, these disorders combined are associated with a 2-4-fold increase in risk of developing cardiovascular disease and all-cause mortality.

Hypertension affects 1 in 3 adults worldwide and is associated with increased risk of cardiovascular disease. The International Diabetes Federation estimates that 425 million people worldwide, or 8.8% of adults, have diabetes with more than 90% of those diagnosed with Type 2 diabetes mellitus (“T2DM”). The concurrent presence of these disorders incrementally increases the risk of developing cardiovascular disease, which is the leading cause of death globally and affects nearly 50% of U.S. adults.

Prevalence of hypertension is expected to increase by more than 9%, or 27 million additional people, from 2010 to 2030 with annual healthcare costs of about $ 131 billion in the United States. Current projections estimate that by 2045 over 629 million people between 20-79 years of age will have T2DM. Current global costs for treating diabetes exceed $727 billion per year or one out of every eight dollars spent on healthcare. While there are multiple pharmacological agents available for treating the symptoms of such conditions, the lack of effective therapies that can slow down or stop the progression for these disorders makes it an urgent public health problem.

Essential or primary hypertension is currently treated with lifestyle modifications or pharmacotherapy. The initial treatment course for hypertension is lifestyle modifications. An important factor with pharmacological treatments is patient non-adherence with prescribed medication. While the exact reasons for non-adherence remain obscure, patient preference inconvenience of life-long therapy, pill holidays, side effects of medication and other factors are important contributors resulting in so called pseudo resistant hypertension, which is reported in >50% of patients. In view of these high rates of non-adherence, it is perhaps not surprising that control rates of hypertension are stagnating and the problem is unlikely to be solved by any additional new medication becoming available.

Similar to hypertension, during early stages of Type 2 diabetes mellitus, the initial course of treatment for most patients includes attention to lifestyle factors (diet, weight loss, and exercise) either alone, or in combination with pharmacologic treatment. Similar to hypertension medications, intolerance and adverse side effects are common with all anti-diabetic pharmacotherapy which includes weight gain, hypoglycemia, gastrointestinal distress, pancreatitis, vitamin B12 and folic acid deficiency, anemia, neuropathy, bone fractures, increase in LDL cholesterol, and diabetic ketoacidosis. As a result of poor adherence and ineffectiveness of drugs to treat underlying causes, most Type 2 diabetes mellitus patients do not reach normal plasma glucose levels. The portion of the diabetic population considered well controlled is low and has decreased in recent years. Eventually, most patients require treatment intensification and progress into complex drug regimens with multiple daily injections including insulin therapy which leads to more risk associated with hypoglycemia and, if severe, can lead to neurological deficits putting patients at increased risk of all-cause mortality.

There is a clear and growing need to effectively address the global epidemic of these diseases that have significant impacts on all-cause and cardiovascular morbidity and mortality. While lifestyle modification and pharmacotherapy can be an effective means to improve these disease states, non-adherence with both treatment strategies is exceedingly common and limits their effectiveness.

### SUMMARY

Systems, devices, and methods described herein advantageously facilitate formation of lesions having a controlled lesion geometry so as to form a single, unitary lesion in a target perivascular space sufficient to functionally denervate an organ (e.g., liver, kidney, pancreas, spleen, small intestine or portion thereof, stomach, etc.). The single, unitary lesion may be generated using a catheter or other medical device advanced to a target intravascular treatment location. The catheter includes multiple energy delivery elements (e.g., electrodes, transducers, or the like or combinations thereof) positioned, controlled, and cooled so as to facilitate the controlled lesion geometry. The systems, devices, and methods may advantageously provide uniform denervation of the perivascular space even though the perivascular tissue adjacent each of the energy delivery elements (also referred to as energy deliver members) may have different perivascular tissue properties.

In accordance with several examples, ablation zones (or lesion geometry) generated by each energy delivery element are blended into a continuous unitary lesion having a target depth, length, and annularity (e.g., circumferentiality) within the perivascular space surrounding a target vessel sufficient to denervate an organ. The ablation zones or lesion geometry generated by each energy delivery element may be generated and controlled so that adjacent ablation zones overlap adjacent ablation zones such that the completed lesion that is formed is uninterrupted without gaps or discontinuities.

The energy delivery elements may be geometrically positioned with both circumferential and axial separation such that the combination of the lesions formed from each energy delivery element results in a single, continuous lesion. During at least part of the energy delivery, the energy delivery elements may be cooled by continuous flow of an externally-delivered coolant circulating within the catheter. The coolant may be provided to an inner surface of the energy delivery elements through an opening in the form of a jet, thereby producing a thermodynamic benefit in which the hottest temperatures associated with the energy delivery are some distance away from the energy delivery elements instead of right at the surface of the energy delivery elements.

In accordance with several examples, a system comprises closed circuit cooling provided by a catheter that includes an inner expandable member and an outer expandable member surrounding the inner expandable member. The inner expandable member is adapted to provide cooling jets to be perfused directly onto respective electrodes of an electrode array of the outer expandable member, thereby providing thermodynamic efficiencies that defer peak electrode temperature away from a vessel wall in which the catheter is positioned and activated. The electrode array includes multiple electrodes that are positioned and integrated so as to allow for a unitary or blended lesion when the procedure is completed. The electrodes are controlled by a power modulation algorithm executed by a generator controller (e.g., including one or more processors, or processing circuitry, as discussed in connection with FIG. 1B) that executes program instructions stored in memory of the generator. The electrodes are electrically connected and the generator adjusts power to each electrode (e.g., voltage and/or duty cycle time are adjusted) in real time based on the tissue properties surrounding each electrode in the perivascular space to ensure the lesion morphology is optimally uniform in depth and annularity.

The generator may perform real-time monitoring of temperature and impedance with predetermined threshold limits such that when the threshold limits are violated, the generator may automatically terminate an ablation cycle (e.g., terminate application of radiofrequency power or energy by the generator to the electrodes of the catheter).

In accordance with several implementations, a method of denervating an organ (or multiple organs) by generating a continuous unitary lesion in a target perivascular space surrounding a vessel (or multiple vessels) at a target intravascular treatment location (or multiple locations) using an intravascular catheter including a distal expandable assembly including one or more electrodes is provided. The method includes positioning the distal expandable assembly of the intravascular catheter including the one or more electrodes at the target intravascular treatment location. The method also includes applying power to the electrodes. The power applied to each of the electrodes is independently modulated (e.g., controlled, adjusted) based, at least in part, on at least one tissue property or characteristic (e.g., tissue impedance and/or temperature) of perivascular tissue surrounding the electrodes. The method also includes causing coolant to be continuously circulated through the distal expandable assembly so as to cool at least one of the electrodes or all of the electrodes. The electrodes are geometrically positioned with both circumferential and axial separation along the distal expandable assembly such that a combination of ablation zones formed by each of the electrodes results in the continuous unitary lesion.

In some implementations, the continuous unitary lesion has a target depth, a target length, and/or a target annularity within the target perivascular space.

The target depth may be between 4 millimeters (mm) and 9 mm beyond (e.g., external to) a wall (e.g., inner wall or outer wall) of the vessel. The target depth may be between 3 mm and 6 mm, between 4 mm and 6 mm, between 5 mm and 8 mm, between 4 mm and 7 mm, between 6 mm and 9 mm, overlapping ranges thereof, or any value within the recited ranges.

The target length may be between 0.5 cm and 1.5 cm. The target length may be between 0.5 cm and 2.5 cm, between 1 cm and 3 cm, between 0.75 cm and 1.5 cm, between 0.5 cm and 4 cm, between 2 cm and 4 cm, overlapping ranges thereof, or any value within the recited ranges.

In several implementations, the target annularity, or circumferentiality, is 360-degree annularity.

The organ or organs to be denervated may include, for example, liver, kidney, spleen, pancreas, small intestine or portion thereof (e.g., duodenum, jejunum), and/or stomach.

In some configurations, the plurality of electrodes are arranged in a 2×2 electrode pattern, wherein a first two electrodes are aligned axially but offset circumferentially, wherein a second two electrodes are aligned axially with each other but offset axially and circumferentially from the first two electrodes, and wherein the second two electrodes are offset circumferentially from each other.

In some configurations, the distal expandable assembly includes an outer expandable member and an inner expandable member. The plurality of electrodes may be positioned along the outer expandable member (e.g., in the 2×2 electrode pattern).

In some implementations, causing the coolant to be continuously circulated through the distal expandable assembly causes the inner expandable member and the outer expandable member to expand and remain expanded, e.g., to place electrodes or other energy delivery elements of the catheter in apposition to a vessel wall. Expansion of the inner expandable member with fluid may cause expansion of the outer expandable member.

In some examples, the inner expandable member includes openings (e.g., outlets, ports, orifices) positioned adjacent locations of each of the plurality of electrodes that are configured to direct jets of the coolant toward each of the plurality of electrodes so as to cool the plurality of electrodes, thereby causing a hottest temperature of the ablation zones to be at locations at a distance from a surface of each of the plurality of electrodes. For example, the openings can each be radially, circumferentially, and/or axially aligned with at least one electrode or other energy delivery element.

In accordance with several implementations, a method of controlling lesion geometry in a target perivascular space surrounding a vessel at a target intravascular treatment location using an intravascular catheter including multiple energy delivery elements so as to provide uniform denervation of the perivascular space even though the perivascular tissue adjacent each of the energy delivery elements may have at least one different tissue property or characteristic that affects energy delivery is provided. The method includes applying a target average power level to each of the energy delivery elements, sensing an actual power level being applied to each of the energy delivery elements, adjusting a duty cycle of each of the energy delivery elements based on the sensed actual power level for the respective energy delivery element; and repeating these steps at a periodic time interval.

In some implementations, applying a target average power level includes applying a target voltage level using a single voltage source.

The target average power level may be between 5 Watts and 10 Watts (e.g., 5 Watts, 6 Watts, 7 Watts, 8 Watts, 9 Watts, or 10 Watts).

The energy delivery elements may include electrodes configured to act as monopolar electrodes and/or ultrasound transducers.

The periodic time interval may be any interval as desired or required, including 0.5 seconds to 10 seconds (e.g., 0.5 seconds, 1 second, 1.5 seconds, 2 seconds, 2.5 seconds, 3 seconds, 3.5 seconds, 4 seconds, 4.5 seconds, 5 seconds, 5.5 seconds, 6 seconds, 6.5 seconds, 7 seconds, 7.5 seconds, 8 seconds, 8.5 seconds, 9 seconds, 9.5 seconds, or 10 seconds).

In some implementations, sensing the actual power level and adjusting the duty cycle is performed in increments (e.g., 5 ms increments, 10 ms increments, 15 ms increments or other increments).

In some implementations, sensing the actual power level includes sensing voltage and current levels for each of the energy delivery elements.

In some implementations, each of the energy delivery elements includes a sensing lead.

The energy delivery elements may be spaced apart from each other axially and/or circumferentially.

In some implementations, the method further includes determining a perivascular tissue impedance for each of the energy delivery elements.

The vessel may be, for example, a renal artery or a hepatic artery (e.g., common hepatic artery).

In some implementations, the energy delivery elements include a plurality of electrodes that are geometrically positioned with both radial, or circumferential, and axial separation along the intravascular catheter.

In some implementations, the method further includes continuously cooling the plurality of energy delivery elements during the method.

In accordance with several implementations, the method results in lesion geometry that is blended into a continuous unitary lesion having a target depth, a target length, and a target annularity.

The target depth may be between 4 mm and 9 mm. The target length may be between 0.5 cm and 2 cm. The target annularity may be 360 degree annularity, or circumferentiality.

In accordance with several implementations, a method of controlling delivery of power to a target perivascular space surrounding a vessel at a target intravascular treatment location using an intravascular catheter including multiple electrodes so as to provide uniform denervation of the perivascular space even though the perivascular tissue adjacent each of the electrodes may have at least one different tissue property or characteristic that affects power delivery (e.g., tissue impedance) is provided. The method includes applying a target average power level to each of the electrodes positioned in contact with an inner wall of the vessel, receiving feedback regarding an actual power level being applied to each of the electrodes, adjusting a duty cycle of each of the electrodes based on the feedback, and repeating these steps (e.g., at a periodic time interval).

In some implementations, the method includes applying a target average power level comprises applying a target voltage level using a single voltage source.

The target average power level may be between 5 and 10 Watts (e.g., 5 Watts, 6 Watts, 7 Watts, 8 Watts, 9 Watts, 10 Watts).

Receiving feedback regarding the actual power level and adjusting the duty cycle is performed at various increments (e.g., 10 ms increments). Receiving feedback regarding the actual power level may include sensing voltage and current levels for each of the energy delivery elements.

In accordance with several implementations, a method of performing renal denervation includes inserting a neuromodulation catheter within a renal artery and advancing a distal expandable assembly of the neuromodulation catheter to a target treatment location within the renal artery. The distal expandable assembly includes an outer expandable structure comprising a plurality of spaced-apart electrodes configured to function as monopolar electrodes. The method also includes causing the distal expandable assembly to transition to an expanded configuration in which the plurality of spaced-apart electrodes are configured to contact an inner wall of the renal artery at spaced-apart locations. The method further includes applying a target average power level to each of the electrodes. The method also includes receiving feedback regarding an actual power level being applied to each of the electrodes and adjusting a duty cycle of each of the electrodes based on the feedback such that each of the electrodes deliver a substantially equal amount of power over a total duration of a treatment procedure such that ablation zones formed adjacent to each of the electrodes overlap and blend to form a single blended, circumferential lesion in a perivascular space surrounding the target treatment location within the renal artery.

In some implementations, applying a target average power level includes applying a target voltage level using a single voltage source. The target average power level may be between 5 and 10 Watts (e.g., 5 Watts, 6 Watts, 7 Watts, 8 Watts, 9 Watts, 10 Watts).

In some implementations, the electrodes are spaced apart from each other axially and/or circumferentially.

In some implementations, the total duration of the treatment procedure is between 90 seconds and 150 seconds

In accordance with several implementations, a method of performing hepatic denervation includes inserting a neuromodulation catheter within a hepatic artery and advancing a distal expandable assembly of the neuromodulation catheter to a target treatment location within the hepatic artery, causing the distal expandable assembly to transition to an expanded configuration in which the plurality of spaced-apart electrodes are configured to contact an inner wall of the hepatic artery at spaced-apart locations, applying a target average power level to each of the electrodes, receiving feedback regarding an actual power level being applied to each of the electrodes, and adjusting a duty cycle of each of the electrodes based on the feedback such that each of the electrodes deliver a substantially equal amount of power over a total duration of a treatment procedure such that ablation zones formed at the spaced-apart locations overlap and blend to form a single blended, circumferential lesion in a perivascular space surrounding the target treatment location within the hepatic artery.

In some implementations, the distal expandable assembly comprises an outer expandable structure including a plurality of spaced-apart electrodes configured to function as monopolar electrodes.

In some implementations, applying a target average power level includes applying a target voltage level using a single voltage source.

The target average power level may be between 5 and 10 Watts (e.g., 5 Watts, 6 Watts, 7 Watts, 8 Watts, 9 Watts, 10 Watts).

In some implementations, the electrodes are spaced apart from each other axially and/or circumferentially.

In some implementations, the total duration of the treatment procedure is between 120 seconds and 180 seconds.

In some examples, the expandable member(s) comprise an inflatable non-compliant bladder. For example, the expandable member(s) may be comprised of a material configured to be non-compliant when enclosed and exposed to internal pressure. In some examples, the material may be configured to be compliant. The expandable support member may comprise a panel (e.g., structure, assembly) formed of multiple layers. A first layer may be a flexible, polymeric base layer and a second layer may be a metallic layer. The layers may comprise sheets or laminates. The polymeric base layer may include one or more perforations extending there through to facilitate access to adjacent layers or sheets. The metallic layer may include one or more conductive electrodes formed in the layer by removing portions of the metallic layer. The electrodes are optimized and positioned to providing both internal exposure to coolant fluid and external exposure to tissue to facilitate high thermal transfer.

The catheter may further include one or more electrical leads (e.g., thermocouple wires) coupled to the one or more conductive electrodes. The electrical leads may be configured to apply power to the electrodes from a radiofrequency generator and to measure temperature and provide temperature information to the radiofrequency generator.

In some examples, the elongate shaft includes multiple lumens. The lumens may be bounded within a reinforcement sleeve that may or may not include an adhesive material therein. The multiple lumens may include a fluid inlet lumen and a fluid outlet lumen fluidly coupled to an external fluid circulation (e.g., delivery and removal) system. The lumens may also include a guidewire lumen configured to receive a guidewire to facilitate tracking of the catheter over a guidewire. The catheter may further comprise a catheter hub assembly comprising multiple ports, each coupled to a respective one of the multiple lumens. The ports may include a fluid inlet port, a fluid outlet port, an electrical port, and/or a guidewire inlet port.

In some examples, the folded waist regions comprise multiple folds or wings that are configured to be folded in a bi-fold, quad-fold, tri-fold, eight-fold, or random-fold configuration. The catheter may further include a collar or sleeve covering the folded waist region of the proximal end portion and/or the folded waist region of the distal end portion of the expandable member, or support structure. In some examples, folds of the folded waist regions are configured to overlap with each other and be joined (e.g., bonded to each other with adhesive, welding, heat bonding, solvent bonding, polymer casting, molding, dip coating, material precipitation or addition, layer by layer solvent formation using polyimide) so as to reduce overall outer delivery profile and prevent fluid leakage. The folded waist regions may be joined (e.g., bonded) to the elongate shaft. The joining techniques may be applied to both the folded waist regions.

In some examples, the at least one metal sheet comprises a copper laminate. The electrical lead wires may comprise multi-filar thermocouple wires configured to apply power and measure temperature (e.g., T-type thermocouple wires). The step of removing portions of the at least one metal sheet may include one or more or etching, photolithography, laser, machining and/or grinding processes. The step of removing portions of the flexible polymeric sheet may include one or more laser cutting, laser stripping, machining, grinding, chemical etching, and/or photo etching processes.

In accordance with several examples, a catheter including a distal expandable electrode assembly comprises or consists essentially of an elongate shaft and an expandable support structure originating as a flat panel or sheet of layers. The expandable support structure includes multiple electrodes formed in the flat panel while the flat panel is in a flat configuration.

In accordance with several examples, a catheter including a distal expandable electrode assembly comprises or consists essentially of an elongate shaft and an expandable support structure originating as a flat panel, the expandable support structure including at least one electrode formed in the flat panel while the flat panel is in a flat configuration. The expandable support structure advantageously enables exterior (e.g., tissue contact) and interior exposure (e.g., internal coolant or fluid that is not released into vessel or body lumen or passage) of the at least one electrode to maximize heat transfer efficiency.

In accordance with several examples, a catheter including a distal expandable electrode assembly comprises or consists essentially of an elongate shaft and an expandable support structure originating as a flat panel, the expandable support structure including at least one thin electrode The expandable support structure may advantageously enable low thermal mass, thereby aiding in a steep thermal gradient for heat removal.

The disclosure relates generally to devices, systems and methods for therapeutically effecting neuromodulation (e.g., hepatic denervation and/or renal denervation) of targeted nerve fibers innervating various organs (for example, the liver, kidneys, pancreas, and/or small intestine) to treat one or more diseases or conditions (e.g., diabetes mellitus, fatty liver conditions or factors of metabolic syndrome such as hyperlipidemia, obesity, and high blood pressure or hypertension). Additionally, neuromodulation of nerve fibers may provide pain relief for patients suffering from abdominal tumors, growths, and or cancers of the liver, kidneys, pancreas, stomach, and/or intestines. In some examples, denervation may include various combinations of organs. In accordance with several examples of the disclosure, disruption of sympathetic nerve fibers innervating the liver is effective to reduce endogenous glucose production and increase hepatic and peripheral glucose storage. The liver is innervated along the structures of the portal triad, particularly the hepatic artery, along which both sympathetic and parasympathetic nerve fibers may course. The nature of the neuroanatomy in this region (e.g., the proximity of neural structures to the arterial lumens of the hepatic arteries such as the common hepatic artery and the proper hepatic artery) is amenable to endovascular approaches for disrupting sympathetic nervous activity, including but not limited to intravascular ablation. Alternative approaches include laparoscopic and/or open surgical procedures, or combinations of such.

Further disclosed herein are expandable (e.g., inflatable by fluid) structures incorporating multiple electrodes for optimal circumferential denervation, wherein catheters incorporating such expandable structures may be used to ablate nerves in a perivascular space surrounding a vessel, e.g., to effect denervation of at least a portion of the liver, kidneys, pancreas and/or duodenum.

For purposes of summarizing the disclosure, certain aspects, advantages, and novel features have been described herein. It is to be understood that not necessarily all such advantages may be achieved in accordance with any particular example disclosed herein.

Thus, the examples disclosed herein may be embodied or carried out in a manner that achieves or optimizes one advantage or group of advantages as taught or suggested herein without necessarily achieving other advantages as may be taught or suggested herein.

### BRIEF DESCRIPTION OF THE DRAWINGS

FIG. 1A schematically illustrates components of an example of a neuromodulation system (e.g., intravascular radiofrequency denervation system).

FIG. 1B is a schematic and conceptual block diagram of a system including an example generator coupled to an example catheter.

FIG. 2 illustrates an example of a neuromodulation (e.g., ablation) catheter of the system of FIG. 1A.

FIG. 3A is a perspective, transparent view of an example of an outer expandable member of a distal expandable assembly of the neuromodulation catheter of FIG. 2.

FIG. 3B is a distal end view of the outer expandable member of FIG. 3A showing an example of an arrangement of the electrodes in four different quadrants around a circumference of the outer expandable member.

FIG. 3C is a side view of the distal expandable assembly of the neuromodulation catheter of FIG. 2 in which portions of the outer expandable member are transparent so that elements and features of the inner expandable member can be viewed.

FIG. 3D is a perspective view of an example of the inner expandable member of the distal expandable assembly of the neuromodulation catheter of FIG. 2.

FIG. 4 is a perspective view of an example of an outer expandable electrode assembly.

FIG. 5 is a side cross-sectional view through an example of a wrapped and folded outer expandable electrode assembly.

FIGS. 6A and 6B are end and side views, respectively, of an example of an inner expandable member (e.g., inner jet member) of the distal expandable assembly having an eccentric proximal waist.

FIG. 7A is a cross sectional view through an example of an elongate shaft of the neuromodulation catheter of FIG. 2.

FIG. 7B is a cross sectional view through an example of a proximal waist of the inner expandable member of FIGS. 3C, 3D, 6A, and 6B.

FIG. 8 illustrates an example of a catheter hub assembly of the neuromodulation catheter of FIG. 2.

FIG. 9A illustrates insertion of the distal expandable assembly of the neuromodulation catheter within a vessel in an unexpanded (e.g., deflated) configuration.

FIG. 9B illustrates the distal expandable assembly of the neuromodulation catheter within the vessel after transitioning to an expanded (e.g., inflated) configuration.

FIG. 10 is a cross-section view that schematically illustrates an example distribution of nerve fibers in a perivascular space surrounding an artery lumen.

FIG. 11 schematically illustrates formation of a single, blended lesion in the perivascular space surrounding an artery lumen that is formed by the neuromodulation catheter.

FIG. 12 is a flow chart illustrating an example power modulation process to control application of power to each of the electrodes of the neuromodulation catheter.

FIG. 13 is a graph illustrating instantaneous power applied to each of the electrodes of the neuromodulation catheter during a portion of an example treatment procedure, as a result of the power modulation process.

FIGS. 14A, 14B, 14C, 14D and 14E show various stages of the formation of a single, blended ablation lesion generated by a neuromodulation catheter and a power modulation process executed by a generator.

FIG. 15 illustrates various example intravascular locations into which the neuromodulation catheter may be inserted and various example organs that may be denervated by the neuromodulation catheter.

FIG. 16A illustrates the anatomy of possible target intravascular treatment locations.

FIG. 16B illustrates various arteries supplying blood to the liver and its surrounding organs and tissues and nerves that innervate the liver and its surrounding organs and tissues.

### DETAILED DESCRIPTION

### I. Introduction and System

In accordance with several examples, the devices and systems described herein are configured for, or designed and adapted for, therapeutic neuromodulation of targeted nerve fibers to treat, or reduce the risk of occurrence of, various diseases, conditions, or disorders, including but not limited to diabetes (e.g., diabetes mellitus), prediabetes, hypertension, metabolic syndrome, sexual dysfunction, and any of the conditions thereof (including hyperglycemia, hypertension, obesity, etc.), nonalcoholic fatty liver disease (NAFLD) and/or nonalcoholic steatohepatitis (NASH). The devices, systems and methods described herein may be used to denervate one or more organs, including, for example, one or more kidneys, the liver, the pancreas, and/or duodenum.

In some examples, the device includes a neuromodulation catheter (e.g., ablation catheter or denervation catheter) including a distal expandable assembly including one or more energy delivery elements (electrodes, ultrasound transducers) and a fluid circulation path to facilitate cooling of the energy delivery elements. The distal expandable assembly may include an expandable support assembly or structure (e.g., inflatable member or balloon-like member, or an expandable basket) for minimally invasive medical procedures. In some examples, the expandable support assembly or structure advantageously provides a thin wall, high strength, and means for attachment to a catheter shaft. Some applications (such as an ablation catheter) may advantageously provide high heat transfer coefficient and/or the ability to include (e.g., affix, attach, couple, form) components and material layers to the support assembly or structure.

In accordance with several examples, the expandable support assembly or structure is advantageously constructed from a flat sheet or panel rolled into a generally cylindrical shape. Cones and waists may be provided by wrapping, folding and/or bonding to facilitate joining the expandable support assembly or structure to a catheter shaft without any leakage or pressure loss through the expandable support assembly or structure. Components, materials, and other features can advantageously be applied to both inner and outer surfaces of the sheet or panel while it is in a flat condition, thereby greatly facilitating the manufacturing process and permitting the use of material available in sheet form (e.g., non-compliant material, polyimide, silicone).

While the description sets forth specific details in various examples, it will be appreciated that the description is illustrative only and should not be construed in any way as limiting the disclosure. Furthermore, various applications of the disclosed examples, and modifications thereto, which may occur to those who are skilled in the art, are also encompassed by the general concepts described herein.

Several examples described herein are aimed to meet several unmet needs involved in intravascular catheter delivery of energy to effect perivascular neuroablation or other neuromodulation including: (i) delivering sufficient energy to achieve a desired single continuous ablation lesion of a certain depth from a vessel wall at the end of the procedure that results in ablation of a clinically effective number of nerves innervating an organ (e.g., a kidney, liver, pancreas, small intestine, stomach, spleen); (ii) limiting the temperature of tissue proximate energy delivery elements (e.g., electrodes) to an isotherm (e.g., isotherm of between 50 and 90 degrees Celsius) to avoid boiling, desiccation and/or arcing; (iii) distributing the energy to achieve circumferential or substantially circumferential perivascular ablation, (iv) limiting adverse impacts to the vessel wall in which the device is positioned and activated to avoid vascular complications, (v) traversing tortuous vascular anatomy, (vi) limiting the size of vessel access puncture; (vii) mitigating the effects of anatomic and physiological variability on the therapy (such as lesion uniformity including annularity, depth and length); (viii) providing the catheter with sufficient mechanical integrity; (ix) providing the catheter with sufficient electrical properties; (x) reduced radiation and contrast dye exposure; (xi) minimal or no leakage of fluid out of the catheter; (xii) minimal or no loss of inflation pressure within inflatable components of catheter; (xiii) ensuring that the catheter is sterilizable, biocompatible and has a practical shelf life; (xiv) assessment of tissue characteristics and treatment effects; (xv) active cooling circuit to control temperature, including the ability to control balance of heat and cooling independent of blood flow; (xvi) ability to increase cooling capacity beyond physiological limits of blood flow; and/or (xvii) providing a uniform, consistent, and/or predictable denervation regardless of perivascular tissue variations.

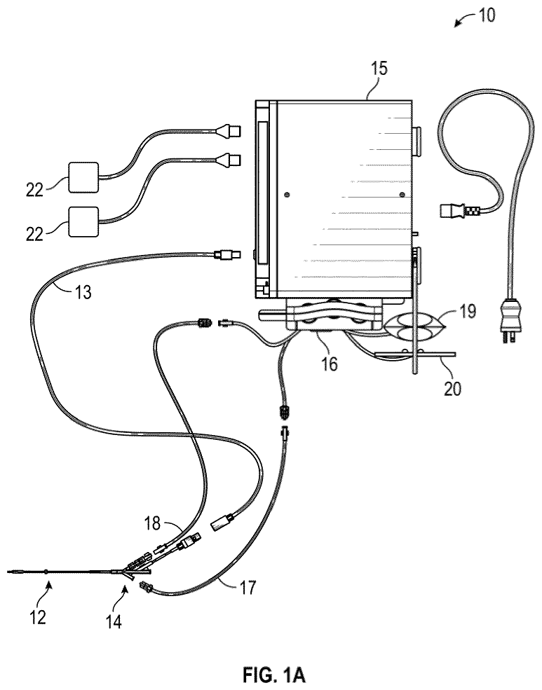

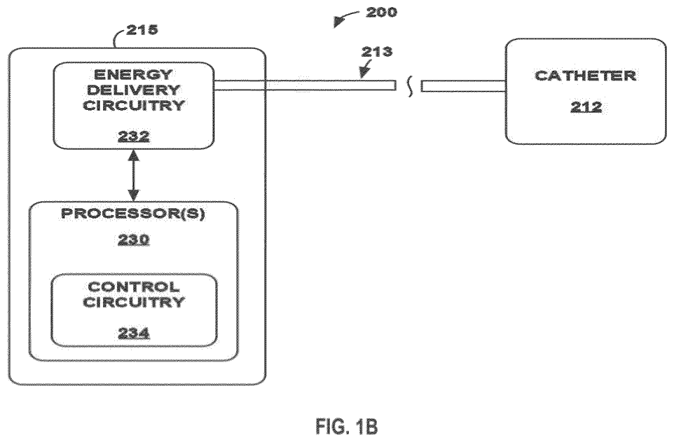

FIG. 1A illustrates an example of a system 10 configured for modulating (e.g., ablating, electroporating, denervating, stimulating) nerves surrounding (e.g., in a perivascular space or area surrounding) a vessel or lumen (e.g., artery or vein) to treat one or more conditions or symptoms associated with the conditions (e.g., metabolic disorders, conditions or factors associated with metabolic syndrome, diabetes, NAFLD, NASH, hypertension, obesity, etc.). The system 10 includes a neuromodulation catheter 12 (e.g., ablation catheter or denervation catheter) including a distal expandable assembly and a multi-lumen shaft. A catheter hub assembly 14 is adapted to be coupled to a proximal end of the shaft of the neuromodulation catheter 12. The catheter hub assembly 14 includes multiple ports, e.g., each port being in fluid communication with and providing access to a lumen of the catheter 12.

The system 10 further includes a generator 15 including power delivery circuitry (e.g., radiofrequency generator) configured to provide power to one or more energy delivery elements of the catheter 12 sufficient to modulate (e.g., ablate) the nerves. A connector cable 13 is provided to establish electrical communication between the catheter 12 and the generator 15. The connector cable 13 may be integrally joined to the catheter 12 or the connector cable 13 may be removably coupled to the catheter 12. The connector cable 13 can have any suitable length. For example, the connector cable 13 may be between 1 foot and 12 feet long, such as about between 30.48 centimeters (cm) and 365.76 cm (e.g., between 1 foot (30.48 cm) and 6 feet (182.88 cm), between 5 feet (152.4 cm) and 8 feet (243.84 cm), between 6 (182.88 cm) and 10 feet (304.8 cm), between 8 feet (243.84 cm) and 12 feet (365.76 cm), overlapping ranges thereof, or any value within the recited ranges). The connector cable 13 may have two conductor leads per electrode. For example, in some examples of catheters 12 having 4 electrodes, the connector cable 13 has at least 8 conductor wire leads: 4 copper wires and 4 constantan wires, providing 4 pairs of T-type thermocouple extension wires. Additional conductors may be provided as desired and/or required. The connector cable 13 terminates in a connector to interface with the generator 15.

In some examples, the generator 15 includes a user interface display. The user interface display may be configured to display output and may optionally receive user input via a touchscreen interface or via one or more user input knobs or buttons. The generator 15 may also be configured to measure temperature via the electrical conductor leads (e.g., thermocouple leads) extending from the generator 15 to the distal expandable assembly (e.g., electrodes, thermocouples, other sensors) through the shaft of the catheter 12.

In some examples, the system 10 also includes a cooling fluid delivery system including one or more fluid reservoirs (e.g., inlet reservoir 19, outlet reservoir 20) to contain the cooling fluid (such as distilled or deionized water or saline solution, physiologic salt solutions, non-ionic colloids such as dextran or glucose, etc.) and one or more pumps 16 (e.g., including one or more syringes or peristaltic pump mechanisms) to effect delivery and/or return of fluid from/to the reservoirs 19, 20. The inlet reservoir 19 may be a sterile water bag (e.g., 1 liter (L) bag). The outlet reservoir 20 may be a bag or other disposable storage container.

In some examples, the cooling fluid delivery system is configured to deliver cooling fluid to the catheter 12 at a controlled (e.g., fixed) flow rate and pressure. For example, cooling may lower (or maintain) the temperature of tissue at below a particular threshold temperature (e.g., at or between 40 to 50 degrees Celsius), thereby preventing or reducing cell necrosis. The catheter 12 is connected to the one or more pumps 16 with fluid connector lines, conduits or tubes that include an inlet extension line 17 and an outlet extension line 18, which are coupled to respective ports of the hub 14 of the catheter 12. The cooling fluid does not necessarily mean that the fluid is cool or cold to touch but just that it is used to cool the electrodes or other energy delivery elements of the catheter 12 as they heat up. For example, the cooling fluid may have a temperature of between 15 and 30 degrees Celsius or may have a temperature similar to room temperature. In some implementations, the cooling fluid may be pre-cooled and may have a temperature lower than 15 degrees Celsius. The inlet extension line 17 and the outlet extension line 18 may be coupled to respective lines or conduits that are operably coupled to the one or more pumps 16. The inlet extension line 17 may be coupled to a corresponding inlet line that is configured to extend through the one or more pumps 16 and to the inlet reservoir 19. The outlet extension line 18 may be coupled to a corresponding outlet line that is configured to extend through the one or more pumps 16 and to the outlet reservoir 20.

In some examples, the cooling fluid delivery is coordinated with the power delivery from the generator 15. Control circuitry of the generator 15, the cooling fluid delivery system, or another device can provide such control. In some examples, there is a delay between the initiation of coolant flow and the initiation of power delivery to allow the coolant flow to reach steady state. Likewise, coolant flow may continue for a time after the cessation of power delivery to avoid thermal injury of the superficial tissues. The control circuitry can terminate power delivery to the energy delivery elements of the catheter 12 if coolant flow faults are detected (e.g., as sensed by one or more flow rate sensors or pressure sensors in the one or more pumps 16 or tubing sections of the fluid delivery system).

The system 10 further can optionally include a protector and stylet assembly configured to cover and protect the distal expandable assembly when in its non-expanded configuration (e.g., while in storage prior to use). The protector forms and maintains the distal expandable assembly (e.g., expandable electrode assembly) in a low profile, wrapped configuration to facilitate passage through a guiding catheter. The stylet may protect the guide wire lumen from collapse or kinking.

FIG. 1B is a schematic and conceptual block diagram of an example system 200 including an example generator 215 and example catheter 212 (which may be coupled via a connector cable 213). The system 200 is an example of the system 10 described in connection with FIG. 1A, and functions described with reference to the generator 215 may be performed by the generator 15 of system 10, with or without other devices. The catheter 212 is an example of the catheter 12 as described in connection with FIG. 1A.

The generator 215 is configured to provide power to the catheter 212 to modulate one or more nerves (e.g., ablate and/or attenuate neural traffic). In some examples, the generator 215 (including energy delivery circuitry 232) may be operatively coupled to or include a power source (e.g., a voltage source, a current source, or another source of energy) configured to deliver energy to the one or more energy delivery elements of the catheter 212. Control circuitry 234 may be configured to control the generator (including energy delivery circuitry 232) to independently modulate power to multiple different energy delivery elements of the catheter 212.

In some examples, the generator 215 includes one or more processors 230 (e.g., control circuitry 234) configured to perform one or more functions related to energy delivery or other therapy delivery as described herein, including control of the delivery of cooling fluid to the catheter 212 in some examples. In general, control circuitry 234 may be configured to perform any of the functions attributed to the generator 215 discussed herein. In some examples, the processors or processing circuitry includes control circuitry 234 (which may also be referred to as a controller 234), are configured to execute one or more stored program instructions and apply denervation therapy.

In some examples, the generator 215 is configured to apply neuromodulation (e.g., ablation) therapy according to one or more parameter values (e.g., as specified by one or more therapy programs). The one or more parameters can include amplitude, duty cycle, frequency, or another suitable parameter or combinations thereof. The control circuitry 234 is configured to control the energy delivery circuitry 232 to generate a neuromodulation therapy signal according to a particular therapy program, and deliver the neuromodulation therapy signal via the catheter 212. The energy delivery circuitry 232 may be electrically coupled to one or more electrical conductors of the catheter 212 using any suitable technique. In some examples, the control circuitry 234 is configured to switch the stimulation generated across different electrodes, or the generator 215 may include multiple energy sources or energy delivery circuits (e.g., radiofrequency generator boards) to drive more than one electrode at one time.

In some examples, the one or more processors 230 is configured to sense one or more parameters (e.g., signals) via energy delivery elements or other sensors of catheter 212. For example, one or more processors 230 may be configured to sense (e.g., receive) instantaneous real-time power actually being delivered to each individual energy delivery element, an impedance (e.g., tissue impedance), or another physiological signal. The one or more processors 230 may be configured to adjust one or more parameters (e.g., a duty cycle) for each energy delivery element based on the sensed parameter (e.g., to achieve a programmed therapy setting). In some examples, generator 215 is configured to measure impedance with reference to a ground electrode 22 or a local bipolar reference electrode.

The one or more processors 230, control circuitry 234, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, the one or more processors 230 and/or the control circuitry 234 includes multiple components, such as any combination of one or more microprocessors, one or more DSPs, one or more ASICs, or one or more FPGAs, as well as other discrete or integrated logic circuitry, and/or analog circuitry.

Although not shown in FIG. 1B, the system 200 (e.g., the generator 215) can also include a memory configured to store program instructions, such as software, which may include one or more program modules, which are executable by the control circuitry 234.

When executed by the control circuitry 234, such program instructions may cause the control circuitry 234 and/or the generator 215 to provide the functionality ascribed to the control circuitry 234 or the generator 215, respectively, herein. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media.

### II. Catheter

### A. Hub

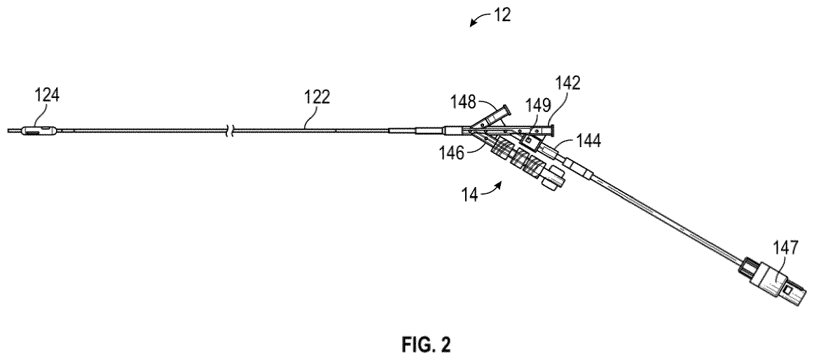

With reference to FIG. 2, the catheter 12 includes a multi-lumen elongate shaft 122, a distal expandable assembly 124, and the catheter hub assembly 14. The catheter hub assembly 14 includes multiple ports. In some examples, the multiple ports include a guidewire port 142, an electrical communication port 144, a fluid inlet port 148, and a fluid outlet port 146. In the example illustrated in FIG. 2, the guidewire port 142 is the in-line axial port and the other ports branch off at angles at various locations. Other arrangements of the ports are possible. The guidewire port 142 is configured to receive a guidewire over which the catheter 12 can be advanced to a target treatment location within a vessel (e.g., renal artery or hepatic artery). The electrical communication port 144 may include a circuit board 149 that includes electrical components and circuitry configured to prevent use of non-proprietary catheters with the generator 15 and may include stored predetermined treatment parameters for the particular catheter 12 that may be transmitted to the generator 15 to control treatment or procedural settings. An interface cable coupled to the electrical communication port 144 may terminate in an electrical connector configured to interface with a respective electrical communication port of the generator 15 or a separate communication cable coupled to an electrical communication port of the generator 15.

### B. Distal Expandable Assembly

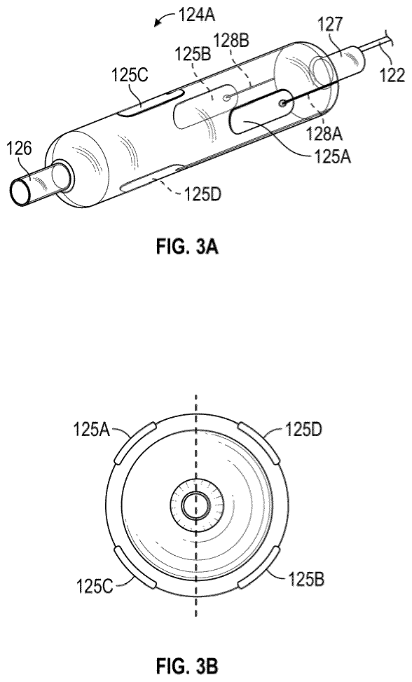

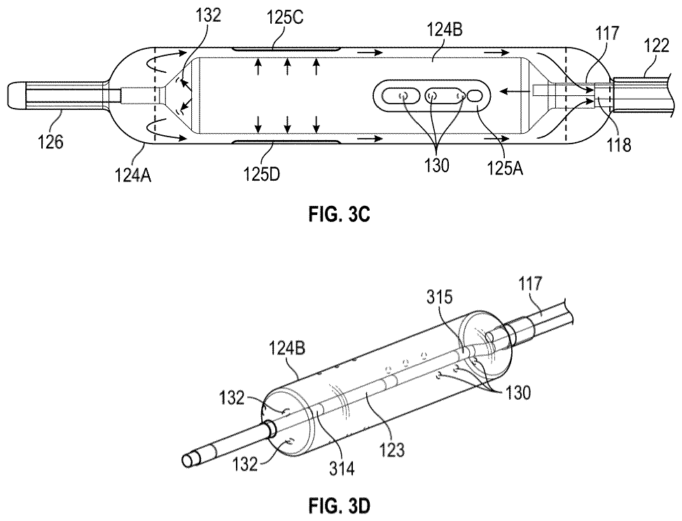

Turning to FIG. 3A, the distal expandable assembly 124 includes an outer expandable member 124A (e.g., electrode support structure). The outer expandable member 124A is configured to transition from a reduced, un-expanded configuration (a relatively low profile configuration) to an expanded configuration (e.g., upon inflation via circulating fluid). The outer expandable member 124A includes multiple electrodes 125A, 125B, 125C, 125D (collectively 125) positioned along its outer surface. As shown, electrodes 125A, 125B are positioned on opposite sides of a circumference of the outer expandable member 124A (e.g., 180 degrees or approximately 180 degrees apart) from each other. Electrodes 125A, 125B are aligned or substantially aligned along an axial distance (e.g., measured along a longitudinal axis of the catheter 12) of the outer expandable member 124A. Electrodes 125C, 125D are positioned on opposite sides of a circumference of the outer expandable member 124A (e.g., 180 degrees or approximately 180 degrees apart) from each other. Electrodes 125C, 125D are aligned or substantially aligned along an axial distance of the outer expandable member 124A. Electrodes 125C, 125D are axially offset (e.g., positioned closer to the distal waist 126) from electrodes 125A, 125B. Electrodes 125C, 125D are also circumferentially offset by 90 degrees or approximately 90 degrees from electrodes 125A, 125B.

Each of the electrodes 125 is electrically coupled to the generator 15 via a respective electrical cable or wire 128A, 128B, 128C, 128D (collectively 128). Each of the electrical cables or wires 128 may include multiple wires (e.g., a constantan/copper pair). The electrical wires 128 may both provide power and be used to measure temperature (e.g., via thermocouple wires). The electrical leads 128 may comprise bifilar, multi-filar or individual lead wires. For example, each electrical lead 128 may include a sensing lead to measure temperature, impedance, and/or cooling rate. The outer expandable member 124A further includes a distal waist 126 and a proximal waist 127.

The end view of FIG. 3B shows how the electrodes 125 may each be positioned on the outer expandable member 124A so as to fall within a separate quadrant around a circumference of the outer expandable member 124A. When in an expanded configuration, or state, the outer expandable member 124A (and thus the electrodes 125) are configured to be positioned in proximate, consistent contact with a vessel wall, substantially or fully occluding blood flow. In accordance with several examples, consistent wall contact and exclusion of blood from the electrode region both advantageously act to reduce biological variability influencing ablation size and temperature.

Turning to FIGS. 3C and 3D, the distal expandable assembly 124 also includes an inner expandable member 124B (e.g., balloon, balloon-like structure, inflatable structure, jet structure). As shown in FIG. 3C, the inner expandable member 124B is nested within the outer expandable member 124A. When cooling fluid is introduced into the inner expandable member 124B via an inlet lumen 117 of the elongate shaft 122, the cooling fluid causes inflation and expansion of the inner expandable member 124B. The inlet lumen 117 is fluidly coupled to an inlet extension line 17 via a fluid inlet port 148 of the hub 14.

In some examples, the inner expandable member 124B includes side outlet ports 130 positioned to align (e.g., radially align), or coincide with, a location of one or more of the electrodes 125 of the outer expandable member 124A, such that cooling fluid may exit the orifices or openings 130 to effect jets of cooling fluid directed at each electrode 125. In some examples, each side outlet port 130 is aligned with (or coincide with) a respective one or more electrodes 125. The jet(s) may advantageously impinge on an interior surface of an electrode 125, thereby providing high velocity gradients and efficient convective heat transfer from the electrode 125 to the tissue surface. In accordance with several implementations, the tissue proximate the luminal surface of the electrode 125 has the highest intensity of RF heating and therefore benefits from more efficient heat transfer. The jets may cool the respective electrode 125 as it is heated during radiofrequency energy delivery during a treatment procedure. In the illustrated example, there are three outlet ports 130 aligned with each electrode 125. However, the number of outlet ports may be less than three or more than three. The inner expandable member 124B may optionally include additional distal outlet ports 132 to facilitate maintenance of circulation of the active cooling circuit and/or to provide additional flow to avoid stasis and to facilitate purging of air bubbles from the catheter 12.

After exiting the inner expandable member 124B and into the outer expandable member 124A through outlet ports 130, 132, the cooling fluid then flows out of the outer expandable member 124A through the outlet lumen 118 extending through the elongate shaft 122. The outlet lumen 118 may be fluidly coupled to the outlet extension line 18 via fluid outlet port 146 of the hub. As shown in FIG. 3D, a guidewire lumen 123 of the elongate shaft 122 extends through the inner expandable member 124B. The cooling fluid that exits into the outer expandable member 124A also inflates or expands the outer expandable member 124A and maintains the outer expandable member 124A in an expanded configuration or state (e.g., maintains sufficient pressure to keep it expanded or inflated).

The outer expandable ember 124A and the inner expandable member 124B can have any suitable size when un-expanded and when expanded. The suitable size may be, for example, selected based on a target blood vessel for the neuromodulation procedure. In some examples, the inner expandable member 124B ranges from about 5 mm to about 100 mm in length (e.g., 20 mm to 50 mm, 10 mm to 30 mm, 5 mm to 50 mm, 50 mm to 100 mm, overlapping ranges thereof, or any value within the recited ranges) and from about 1 mm to about 35 mm in diameter (e.g., 1 mm to 10 mm, 2 mm to 7.5 mm, 5 mm to 15 mm, 15 mm to 35 mm, overlapping ranges thereof, or any value within the recited ranges). The inner expandable member 124B may be provided with a proximal cone and a proximal waist and a distal cone and a distal waist to for attachment to the elongate shaft 122.

The orifices or openings 130 and the auxiliary openings 132 can also have any suitable size and spacing, which can, for example, depend on the desired cooling effect and/or the size of the electrodes 125 or other energy delivery elements. In some examples, the orifices or openings 130 and the auxiliary openings 132 have a diameter of 0.001 inches (in) (0.0254 mm) to 0.010 in (0.254) (e.g., between 0.001 in (0.0254 mm) and 0.005 in (0.127 mm), between 0.002 in (0.0508 mm) and 0.004 in (0.1016 mm), between 0.003 in (0.0762 mm) and 0.007 in (0.1778 mm), between 0.005 in (0.127 mm) and 0.010 in (0.254 mm), overlapping ranges thereof, or any value within the recited ranges). In some examples, the orifices or openings 130 are spaced apart at between 1 mm and 3 mm (e.g., between 1 mm and 2 mm, between 2 mm and 3 mm, between 1 mm and 2.5 mm). In some implementations, the orifices or openings 130 may be spaced apart at a distance of less than 1 mm or greater than 3 mm.

In some examples, the orifices or openings 130 (and the optional auxiliary openings 132) are configured to facilitate a flow rate of between 0.51 mL/sec and 12.0 mL/sec (e.g., between 0.5 mL/sec and 0.75 mL/sec, between 0.7 mL/sec and 0.9 mL/sec, between 0.8 5 mL/sec and 1.0 mL/sec, between 0.1 mL/sec and 1.0 mL/sec, between 1.0 mL/sec and 2.0 mL/sec, overlapping ranges thereof, or any value within the recited ranges). The flow rate may be less than 0.5 mL/sec or greater than 1 mL/sec in certain implementations.

In some examples, the fluid exits the orifices 130 as a high velocity jet. In one example, the flow rate of coolant fluid through an orifice is about 0.1 milliliters per second (mL/s) and the pressure drop across the orifice is about 500 kilopascals (kPa). Jet velocity may range from about 5 meters per second (m/s) to about 50 m/s (e.g., 5 m/s to 30 m/s, 15 m/s to 30 m/s, 15 m/s to 40 m/s, 20 m/s to 40 m/s, 35 m/s to 50 m/s, overlapping ranges thereof, 22 m/s, or any value within the recited ranges. In some examples, the distance from a respective orifice 130 to a respective electrode 125 may range from about 0.10 mm to about 2.0 mm (e.g., between 0.10 mm and 0.50 mm, between 0.50 mm and 1.0 mm, between 0.50 mm and 1.5 mm, between 1.0 mm and 2.0 mm, between 0.10 mm and 1.0 mm, overlapping ranges thereof, or any value within the recited ranges).

In some examples, the outer expandable member 124A is designed to perform with a pressure strength of greater than 30 psi, greater than 100 psi, or other pressure strengths. A pressure strength of between 100 psi and 150 psi has been found to provide adequate factor of safety for both normal operating pressures of 20psi- 40 psi and transient pressure increases as may occur due to catheter obstruction. The compliance of the outer expandable member 124A may range from 0.01 millimeter per atmosphere (mm/atm) to 0.1 mm/atm (e.g., 0.01, 0.02, 0.03. 0.04, 0.05, 0.06, 0.07, 0.08, 0.09, 0.1 mm/atm).

The outer and inner expandable members 124A, 124B can be formed from any suitable material and can have any suitable size. In some examples, the outer expandable member 124A may include a flexible polymer sheet (e.g., polyimide, polyester, Mylar, PTFE, or combinations thereof) that is rolled into a cylinder. The outer expandable member 124A may have a diameter between 3 mm and 35 mm (e.g., between 3 mm and 8 mm, between 5 mm and 8 mm, between 4 and 7 mm, between 6 mm and 12 mm, between 8 mm and 12 mm, between 15 mm and 25 mm, between 20 mm and 35 mm, overlapping ranges thereof, or any value within the recited ranges). The outer expandable member 124A may have a length between 10 mm and 100 mm (e.g., between 10 mm and 25 mm, between 15 mm and 20 mm, between 20 mm and 25 mm, between 25 mm and 75 mm, between 50 mm and 100 mm, overlapping ranges thereof, or any value within the recited ranges).

In accordance with several examples, the outer expandable member 124A is coupled to the shaft 122 by forming folded cones and waists on both the proximal end and the distal end of the outer expandable member 124A. The folded cones and waists 126, 127 form an enclosed volume and prevent fluid leakage out of the outer expandable member 124A. The folds of the proximal and distal ends may comprise a quad fold configuration, an 8-fold configuration, or a random fold configuration. The waists 126, 127 may be coupled (e.g., bonded) to the shaft 122 with adhesive and/or thermoplastic on inner and/or outer surfaces (e.g., a modified acrylic adhesive, such as LF-1500, available from DuPont de Nemours, Inc. of Wilmington, Delaware, 1MP polyurethane, nylon hot melt, and/or the like). The adhesive may be limited to the waist regions 126, 127 or may extend onto the respective cones. A reinforcing sleeve may be applied to the outer surface of one or both of the waists 126, 127.

The electrodes 125 are arranged on the outer expandable member 124A to facilitate the neuromodulation energy delivery. In some examples, the electrode pattern of the outer expandable member 124A comprises an offset staggered electrode pattern. In the illustrated example, the outer expandable member 124A includes four electrodes 125 arranged in a 2×2 pattern, with a first two electrodes positioned 180 degrees apart circumferentially and at the same first axial, or longitudinal, distance or position along the outer expandable member 124A, and a second two electrodes positioned circumferentially opposite (e.g., 180 degrees apart) and at the same second axial, or longitudinal, distance or position along the outer expandable member 124A, with the second axial distance or position being different than the first axial distance or position, and with the second two electrodes positioned angularly offset (e.g., 90 degrees offset circumferentially) from the first two electrodes. This 2×2 offset pattern, as well as other possible electrode patterns that may be used (e.g., spiral patterns, 3 or 4 or more electrodes in the same longitudinal plane but radially spaced apart, and/or multiple clusters of electrodes in a 2×2 offset pattern or a spiral pattern) are described in further detail in U.S. Publication No. 2017/0348049 by Vrba et al. and/or WIPO Publication No. WO 2016/090175 naming inventors Vrba et al., which are incorporated herein by reference. The electrode pattern may advantageously increase the overall perivascular ablation volume while maintaining little to no thermal damage or endothelialization (e.g., less than 20% mean maximum circumference of vessel injury, no internal elastic lamina disruption, no arterial dissection, and/or no clinically significant neointimal formation, no long-term vascular stenosis, no circumferential vessel wall injury) to the portions of the vessel wall in contact with the electrodes 125.

In accordance with several examples, the pattern (including point spacing, electrode size, energy algorithm, circumferential offset) is configured to produce ratios of circumferential perivascular injury to circumferential vessel wall injury of greater than or equal to 2:1 (e.g., 5:1, 4:1, 3:1, 2:1).

In accordance with several examples, the staggered “checkerboard” or offset pattern of electrode placement on the outer expandable member 124A advantageously provides for electrical and thermal interaction of the fields within the tissue, thereby resulting in greater depth and better blending of the lesion, which in turn, provides for more effective neuroablation or other neuromodulation. At the same time, the limited vessel injury “footprint” avoids circumferential vessel wall injury, thereby reducing likelihood of stenosis or other complications. In accordance with several examples, the fraction of vessel circumference that is injured is less than 50% (e.g., less than 45%, less than 40%, less than 35%). When the fraction of vessel circumference injured is less than 50%, vascular complications can likely be avoided.

The electrodes 125 can each have any suitable configuration (e.g., shape, size, material, and/or the like). All the electrodes 125 of the catheter can have the same configuration or two or more electrodes 125 can have different configurations. In accordance with several examples, a length (e.g., longitudinal dimension) of each electrode 125 may range from 2 mm to 10 mm (e.g., 2 mm to 4 mm, 3 mm to 5 mm, 2 mm to 6 mm, 3 mm to 5 mm, 3 mm to 6 mm, 4 mm to 6 mm, 4 mm to 8 mm, 6 mm to 10 mm, overlapping ranges thereof, or any value within the recited ranges). The electrodes 125 may each have a surface area of greater than 1 mm² (e.g., between 1 mm² and 5 mm, between 5 mm² and 10mm², between 8 mm² and 12mm², between 10 mm² and 15mm², between 12 mm² and 20mm², overlapping ranges thereof, or any value within the recited ranges). Electrical energy delivered to the tissue is distributed over the electrode surface with a greater concentration near the edges of the electrodes. Larger electrodes both reduce the power density in the adjacent tissue and provide for greater cooling surface area. Much of the heat is conducted into the surrounding tissue to create the ablation. However, some heat must be removed from the inside of the electrode to prevent overheating. This heat extraction rate is governed by thermal conduction through tissue and the polymer layers of the electrode assembly, as well as convective cooling from the cooling fluid. Heat removal via convective cooling may range from 0.5W-2 W.

In examples where the generator 15 is an RF generator, the therapeutically effective amount of RF energy at the location of the inner vessel wall of the target vessel or at the location of the target nerves is in the range of between about 100 Joules (J) and about 2 kilojoules (kJ) (e.g., between about 100 J and about 1kJ, between about 100 J and about 500 J, between about 250 J and about 750 J, between about 300 J and about 1 kJ, between about 300 J and about 1.5 kJ, between about 500 J and 1 kJ, or overlapping ranges thereof). In one example, the therapeutically effective amount of RF energy has a power between about 0.1 W and about 14 W (e.g., between about 0.1 W and about 10 W, between about 0.5W and about 5 W, between about 3 W and about 8 W, between about 2 W and about 6 W, between about 5 W and about 10 W, between about 8 W and about 12 W, between about 10 W and about 14 W, or overlapping ranges thereof). In some implementations (e.g., for pulmonary vein isolation techniques), the therapeutically effective amount of RF energy has a power greater than 14 W (e.g., from 14 W up to 40 W, such as 20 W, 25 W, 30 W, 35 W, 40 W). The ranges provided herein can be per electrode, per energy delivery location, or total energy delivery.

The RF energy may be delivered at one location or multiple locations along the target vessel or within multiple different vessels. In some examples, the RF, energy is delivered sufficient to cause fibrosis of the tissue surrounding the nerves, thereby resulting in nerve dropout. In some examples, various electrodes 125 along the length of the outer expandable member 124A are toggled on or off to customize treatment length. The electrodes 125 may be configured to act as monopolar electrodes (e.g., in conjunction with one or more ground pads 22, which can be external ground pads in some examples) or as bipolar electrodes.

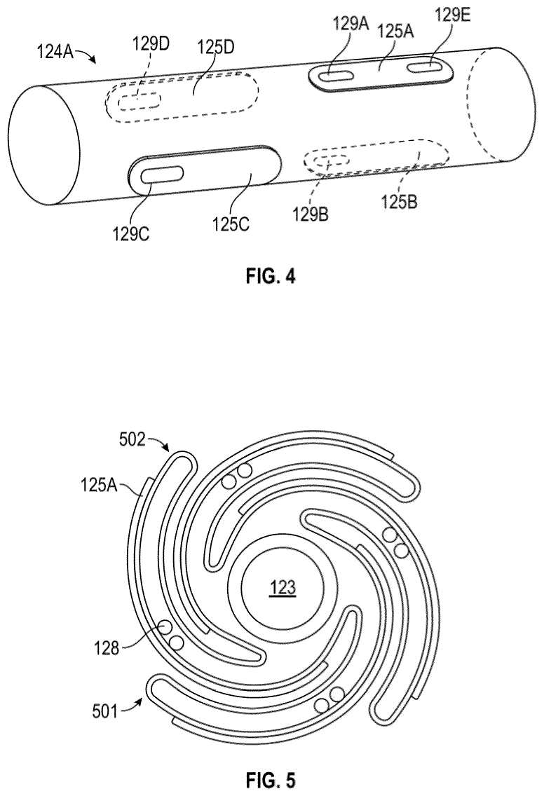

Turning to FIG. 4, in some examples, the electrodes 125 may be comprised of radiopaque material and may also include orientation markers 129A, 129B, 129C, 129D, 129E. In some examples, radiopaque material is applied to one or more electrodes 125 to indicate electrode orientation and visualize expansion. Radiopacity may be provided by solder, for example lead solder. In other examples, radiopacity is provided by applying silver solder or gold solder, selective gold electroplating of the electrodes 125, gold swaged onto one or more of the electrical lead wires 128, gold plating of one or more of the electrical lead wires 128 after being stripped, and/or radiopaque ink. Resolution of angular position from 2-dimensional images can be accomplished through the application of multiple or chiral markers (e.g., right handed or left handed). For example, an index electrode (e.g., electrode 125A) may be identified by a second index marker 129E in addition to the first index marker 129A. A third marker 129C at a different radial position can resolve ambiguity regarding electrode orientation. In the illustrated example, a first electrode 125A is placed circumferentially opposite a second electrode 125B in the same or substantially the same longitudinal position along the outer expandable member 124A. A third electrode 125C is provided at a second longitudinal position at an angular position offset from the first two electrodes 125A, 125B. A fourth electrode 125D is positioned opposite (e.g., radially, or circumferentially) the third electrode 125C.

FIG. 5 is a side cross-sectional view through an example of a wrapped and folded proximal end region of the outer expandable member 124A. The illustrated example shows a quad-folded electrode assembly configuration, although other folding configurations may be used (e.g., 8-fold, bi-fold, tri-fold, random-fold configurations). Folding allows the catheter to be delivered through a smaller profile guide catheter, while the distal expandable assembly is in the folded, unexpanded configuration.

As shown, portions of each respective fold overlap each other. For example, a portion of a first fold 501 overlaps a portion of an underlying second fold 502. In several examples, a width of each electrode 125 is less than a width of its respective fold outer face so as not to interfere with folding and so as to allow a reduced outer profile when in the folded configuration. As shown, each electrode 125 is positioned to coincide with an outer fold face when in the folded configuration. The electrical leads 128 are free floating but may be soldered or otherwise adhered to a respective electrode 125 at a discrete location to preserve flexibility and facilitate routing from the outer expandable member 124A through a lumen (e.g., guidewire lumen 123) of the shaft 122. In some examples, the delivery profile (e.g., maximum outer cross-sectional dimension) may be sized and configured to fit through an 8 French guide catheter when in the wrapped and folded configuration (e.g., between 0.075 in (1.905 mm) and 0.100 in (25.4 mm)).

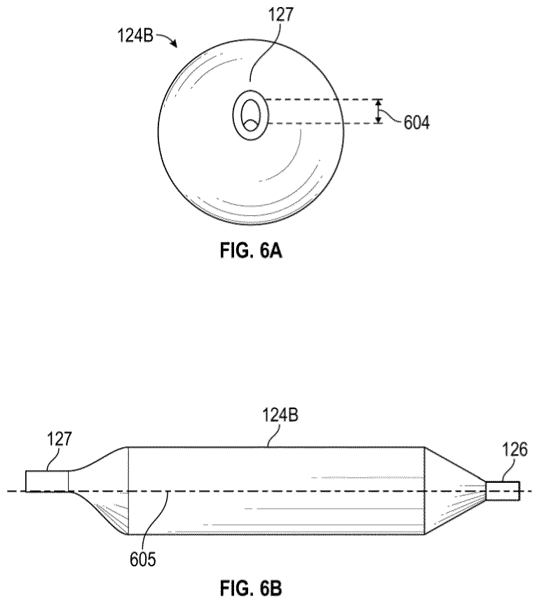

Turning now to FIGS. 6A and 6B, the inner expandable member 124B may optionally exhibit an eccentric configuration, in that a central longitudinal axis 605 of the inner expandable member 124B does not pass through the center of both the proximal waist 127 and the distal waist 126. The proximal waist 127 may be ovalized and offset by a distance 604 from the central longitudinal axis 605 to improve alignment of the jets formed by the orifices or openings 130 of the inner expandable member 124B and the electrodes 125 of the outer expandable member 124A. The fluid inlet lumen 117 may deform or bend to pass through the proximal waist 127.

### C. Elongate Shaft

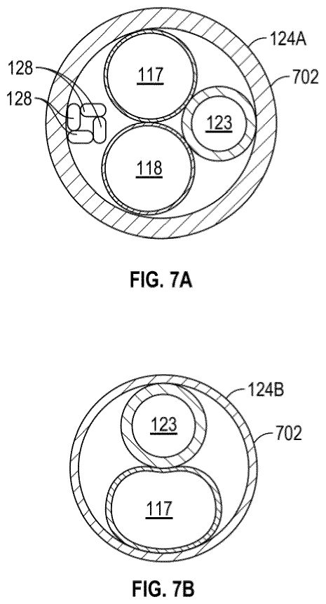

With reference to FIGS. 7A and 7B, in some examples, the multi-lumen shaft 122 is an elongate flexible shaft comprised of a loose bundle of tubes surrounded by a flexible sleeve 702. In some examples, the shaft 122 is between 60 cm and 150 cm long (e.g., between 60 cm and 80 cm, between 70 cm and 90 cm, between 75 cm and 85 cm, between 80 cm and 100 cm between 100 cm and 150 cm, overlapping ranges thereof, or any value within the recited ranges). The outer diameter of the sleeve 702 may be between 0.035″ and 0.090″ (e.g., between 0.035″ and 0.065″, between 0.065″ and 0.090″, between 0.070″ and 0.080″, between 0.075″ and 0.085″, overlapping ranges thereof, or any value within the recited ranges). The sleeve 702 may be formed of any suitable material. In some examples, the sleeve 702 is formed of one or more of the following materials: 72D PEBAX elastomer, 63D PEBAX elastomer, 55D PEBAX elastomer, high-density polyethylene (HDPE), perfluoroalkoxy (PFA), polytetrafluoroethylene (PTFE)). In some examples, the shaft 122 may transition from harder to softer durometer in the a distal direction and/or in a distal region.

FIG. 7A is a cross-sectional view of an example of the shaft 122, the cross-section being taken in a direction orthogonal to a longitudinal axis of the shaft 122. The multi-lumen shaft 122 includes the guidewire lumen 123, the fluid inlet lumen 117 and the fluid outlet lumen 118. Although FIG. 7A shows one arrangement of the various lumens, other arrangements may be used. The electrical lead wires 128 are also shown in FIG. 7A, as they extend through the shaft 122 and into the annular gap between the inner expandable member 124B and the outer expandable member 124A.

FIG. 7B is a cross-sectional view through an example of the proximal waist 127 of the inner expandable member 124B, the cross-section being taken in a direction orthogonal to a longitudinal axis of the shaft 122. As shown, the guidewire lumen 123 and the fluid inlet lumen 117 extend into the inner expandable member 124B, whereas the fluid outlet lumen 118 shown in FIG. 7A does not, as it only extends into the outer expandable member 124A to expel the cooling fluid after it has exited the inner expandable member 124B.

The guidewire lumen 123 may be sized so as to be compatible with a 0.014″ guide wire. Alternatively, the guidewire lumen 123 may be sized so as to be compatible with a 0.018″ or a 0.035″ guidewire. Polyimide and/or PTFE lining or composites may also be used. The fluid inlet lumen 117 and the guidewire lumen 123 may be bonded into the proximal waist 127 of the inner expandable member 124B by adhesive. The fluid inlet lumen 117 may comprise a polyimide tube connecting the fluid inlet port 148 of the cooling fluid system to the inner expandable member 124B.

In some examples, the cooling fluid system and the fluid inlet lumen 117 (via inlet extension line 17) are configured to provide a 0.5 mL/sec to 1.5 mL/sec (e.g., 0.8mL/sec) flow rate at approximately 25psi-35 psi (e.g., 25 psi, 26 psi, 27 psi, 28 psi, 29 psi, 30 psi, 31 psi, 32 psi, 33 psi, 34 psi, 35 psi) pressure drop. The fluid outlet lumen 118 may comprise a polyimide tube connecting a fluid outlet port to the outer expandable member 124A. In some examples, the cooling fluid system and the fluid outlet lumen 118 may be configured to provide a 0.5 mL/sec to 1.5 mL/sec (e.g., 0.8mL/sec) flow rate at approximately 20 psi- 30 psi (e.g., 20psi, 21 psi, 22 psi, 23 psi, 24 psi, 25 psi, 26 psi, 27 psi, 28 psi, 29 psi, 30 psi) pressure drop.

In some examples, the electrical leads 128 may comprise thermocouple wires, such as, but not limited to, 4 pairs of 40 Gauge T-type thermocouple wires in insulation (e.g., nylon, polyimide, polyurethane, PTFE, fluorinated ethylene propylene (FEP), PFA, polyester, and/or the like). Each pair of leads 128 is attached to a respective electrode 125 of the outer expandable member 124A and to corresponding pins on the electrical cable connector 147 attached to the catheter hub assembly 14. In some examples, fluid is excluded from the interstitial spaces within the sleeve 702 to reduce heat transfer into the fluid inlet lumen 117 and reduce RF capacitive leakage to the surrounding tissue. Crosstalk between leads 128 may also be reduced.

In several examples, the loose bundle of tubes is bonded near its distal end within the proximal waist 127 of the outer expandable member 124A. At the proximal end of the bundle of tubes or lumens, the tubes or lumens pass into the catheter hub assembly 14. In some examples, the outer sleeve 702 is butt joined to the proximal waist 127 of the outer expandable member 124A. In some examples, adhesive bonds the sleeve 702 of both the loose bundle of tubes or lumens and reinforces the proximal waist 127 of the outer expandable member 124A.

As shown in FIG. 3D, the shaft 122 (e.g., guidewire lumen 123 portion of the shaft 122) may include marker bands 314, 315. In some examples, the marker bands 314, 315 comprise radiopaque material (e.g. platinum iridium alloy, gold). The markers 314, 315 may be placed on the shaft 122 delineating outer electrode margins to facilitate placement within a vessel. A third marker band may be placed between the electrodes 125 to further highlight the electrode margins.

### D. Catheter Hub Assembly

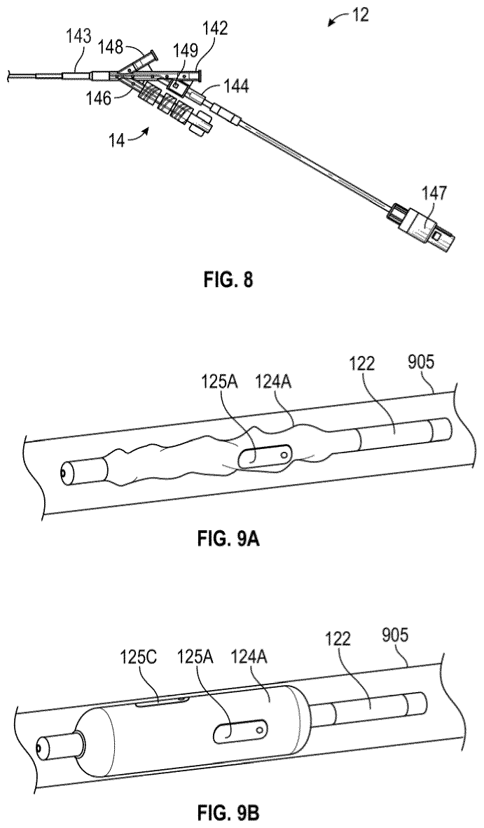

With reference to FIG. 8, the catheter hub assembly 14 includes a strain relief 143 to prevent kinking of the catheter hub assembly 14. The catheter hub assembly 14 is configured to provide access to each of the lumens 123, 117, 118 and lead wires 128 within the shaft 122. The catheter hub assembly 14 includes an electrical communication port 144, a fluid inlet port 148, a fluid outlet port 146, and a guidewire port 142. The guidewire port 142 is configured to facilitate insertion and passage of an intravascular guidewire. The catheter hub assembly 14 further includes or is separate from but coupled to a connector including electrical connections to connect the catheter electrical leads 128 to an extension cable. The connector can include, for example, a cable connector (e.g., cable connector 147).

### III. Methods of Use and Lesion Formation

The catheter 12 is configured for intravascular introduction to a target treatment location. As shown in FIG. 9A, the catheter 12 may be advanced along vasculature with the distal expandable assembly 124 in an unexpanded configuration until the distal expandable assembly 124 is positioned at the target treatment location within a certain vessel 905 (e.g., hepatic artery, renal artery, gastroduodenal artery, splenic artery, mesenteric artery). As shown in FIG. 9B, the distal expandable assembly 124 may then be caused to expand to an expanded configuration (e.g., by activating the cooling fluid delivery system and introducing cooling fluid into the expandable members 124A, 124B) when the distal expandable assembly 124 has been advanced to the target treatment location. The distal expandable assembly 124 may be expanded such that an outer surface of the outer expandable member 124A (including the electrodes 125) are in contact with an inner wall surface of the vessel 905 at the target treatment location.

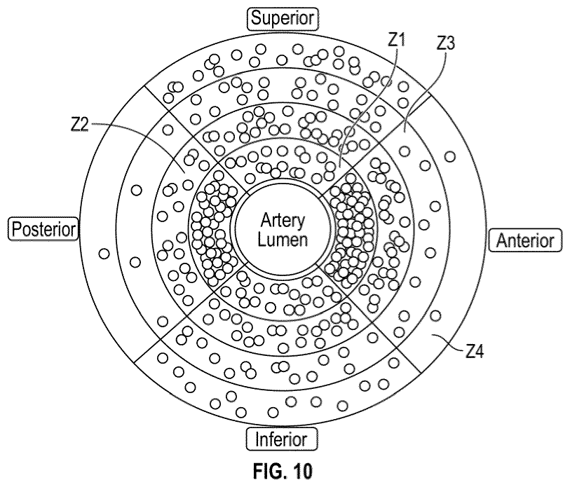

FIG. 10 illustrates an example distribution of nerve fibers in a perivascular space surrounding an artery lumen. As shown, the nerve fibers may fall within one or more concentric zones Z1-Z4 surrounding the artery lumen. The distribution of nerve fibers may be more concentrated in the inner zones Z1 and Z2 than in the outer zones Z3 and Z4. In accordance with several examples, the catheters 12 described herein are advantageously designed so as to effect denervation of the nerve fibers to a certain depth from the artery lumen so as to effectively denervate the nerve fibers in multiple zones of the perivascular space. For example, the catheters 12 may be designed to efficaciously denervate the nerve fibers up to a depth of 4 mm, 5 mm, 6 mm, 7 mm, 8 mm, 9 mm, 10 mm, or more than 10 mm from the artery lumen wall, depending on the particular artery and the expected distribution of nerve fibers surrounding the artery lumen. As another example, the catheters 12 may be designed to efficaciously denervate a certain percentage of the nerve fibers (e.g., 50-75%, 60-80%, 70-90%) in the perivascular space surrounding the artery lumen at the target treatment site.

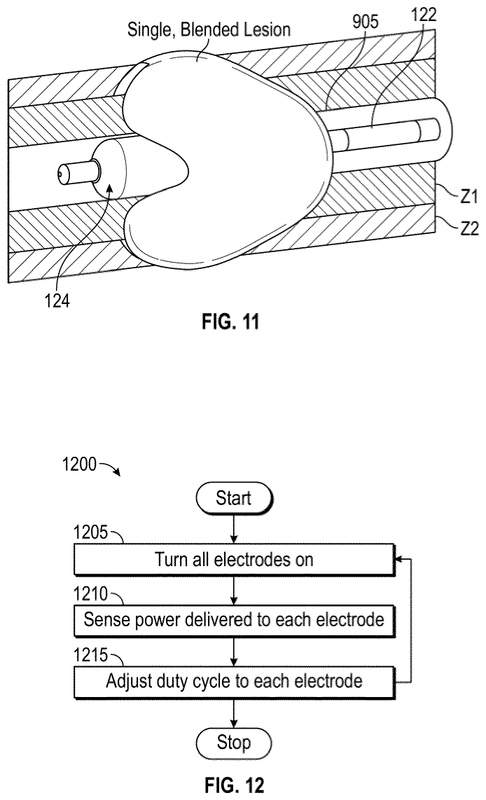

With reference to FIG. 11, the catheter 12 may be controlled and designed so as to form a single, blended lesion upon completion of a treatment procedure. The single, blended lesion provides circumferential ablation (e.g., with 360-degree annularity) to a sufficient depth and having a sufficient length so as to denervate a clinically effective amount of nerve fibers in the perivascular space surrounding the vessel lumen 905 at the target treatment site without requiring advancement to, and treatment at, additional locations within the vessel lumen 905. In some examples, the sufficient length ranges from 0.5 cm to 2 cm (e.g., from 0.5 cm to 1.5 cm, from 1 cm to 1.5 cm, from 1 cm to 2 cm). In some examples, the sufficient length is at least 1 cm.

In accordance with several examples, the single, blended lesion is sufficiently formed such that treatment is clinically effective for a particular vessel (e.g., hepatic artery, renal artery, or another blood vessel) by ablating at only one target treatment site (e.g., only a single lesion is formed at a single treatment site within the vessel such that it is a “one-and-done” treatment procedure within each target vessel). The single, blended lesion may also advantageously be uniform and consistent regardless of perivascular conditions (e.g., tissue variations). Thus, total treatment time and potential vessel injury are reduced by not forming lesions at multiple different spaced-apart treatment sites within a single vessel. Afferent and efferent nerve fibers are both ablated and may not be differentiated or selectively targeted in several examples.

The depth and length and circumferentiality (e.g., 360-degree annularity) of the single, blended lesion that is uniform regardless of perivascular conditions (e.g., due to the heterogeneous nature of tissue surrounding an artery or other lumen) may advantageously be facilitated by control of the power delivery by the control circuitry 234 of the generator 215 (FIG. 1B) alone or in combination with control circuitry of another device (e.g., a cooling delivery system). Although the description primarily refers to the control circuitry 234 in the description of the control of neuromodulation therapy delivery to achieve a single, blended lesion or other lesion, in other examples, control circuitry of another device can be used in addition to or instead of the control circuitry 234 to control the neuromodulation therapy. The control circuitry 234 can execute stored program instructions of a power modulation control algorithm. The program instructions may be stored in memory of the generator 15 or a memory of another device. The program instructions may alternatively be stored on a printed circuit board or chip 149 within the catheter hub assembly 14.

The perivascular space surrounding a particular vessel lumen may be comprised of both local fat and muscular tissue having significantly different electrical and acoustic impedance properties. Energy flows preferentially to lowest impedance tissue and so tissue ablation or denervation systems that do not account for differences in perivascular conditions at different treatment element locations (e.g., electrode locations, transducer locations) can result in uneven treatment depths throughout the treatment circumference, which may affect consistency and reliability in treatment efficacy.

Turning to FIG. 12, a flowchart of an example power modulation control algorithm or process 1200 is illustrated. At step 1205, the control circuitry 234 activates all of the electrodes 125 on the outer expandable member 124A (e.g., turned on by applying power to the electrodes via electrical wires 128 extending between the generator 15 (e.g., a single voltage source of the generator 15) and the electrodes 125). The generator 15 is programmed to apply the same average power to each of the electrodes 125 (e.g., 5 W or 6 W average power) so as to provide a consistent, uniform and reliable denervation procedure regardless of perivascular tissue variations. However, in reality, the actual instantaneous power delivered to each individual electrode 125 may vary as a result of perivascular tissue variations (e.g., tissue impedance differences) adjacent the contact locations of each individual electrode 125. For example, perivascular tissue impedance at one electrode location may be higher than another due to the type of tissue present in the perivascular space at that electrode location. For example, tissue with more fat may have a different impedance than tissue with less fat.

Because power delivery is affected by tissue impedance, for a given amount of power (e.g., a target power level) applied to each electrode by the generator 15, differences in tissue impedance will affect the amount of power actually delivered to each electrode and adjacent tissue, and can lead to uneven treatment depths along the vessel (e.g., renal or hepatic artery). The same effect may be true for ultrasound transducers and differences in acoustic impedance between perivascular tissue at different locations.

In order to achieve the single blended lesion having a desired depth, length and circumferentiality (e.g., 360-degree annularity), the control circuitry 234 modulates the power delivered to each individual electrode 125 so that at the end of the procedure, the same total amount of power is delivered by each electrode to tissue proximate the respective electrode. If the power is not modulated, then some electrodes would deliver more power than others and the lesion shape formed at the end of the procedure may not form a single, blended lesion having the desired depth, length and circumferentiality (e.g., 360-degree annularity) to be clinically efficacious due to the differences in perivascular tissue variation (e.g., tissue impedance differences).

Accordingly, at step 1210, the control circuitry 234 (e.g., upon execution of the stored program instructions) determines (e.g., by sensing or by receiving information) the instantaneous real-time power actually being delivered to each individual electrode 125 (e.g., through current and voltage measurements sensed within the generator 15 and/or based on sensed tissue impedance) and adjusts a duty cycle for each electrode 125 at step 1215 (e.g., in 10 ms increments) so as to achieve the programmed average power setting. As an example, in response to determining the impedance of an electrical pathway including a particular electrode 125 is lower (e.g., by a threshold amount or lower by any amount) than the impedance of the electrical pathways including other electrodes 125, the control circuitry 234 can control the power delivery to the particular electrode 125. The lower impedance can result in more power being delivered to tissue via the particular electrode for a given power level (e.g., a target average power level). Thus, in some examples, the control circuitry 234 controls energy delivery circuitry 232 (FIG. 1B) to pause the power application to the particular electrode or to lower the power being applied to the particular electrode for a certain amount for a particular amount of time (e.g., a preset amount of time, which can change based on the impedance difference in some examples or which can be the same regardless of the impedance difference in other examples). In some of these examples, the control circuitry 234 controls the energy delivery circuitry 232 to deliver the same (or nearly the same to the extent permitted by system tolerances) target power level to each electrode 125 during an ablation procedure. Thus, the total power delivered by each electrode 125 can be modified by modifying the duty cycle (e.g., relatively on/off times for power application to the particular electrode 125). The target power level can be a target voltage level, e.g., such as from a single voltage source for each electrode 125. In some examples, the target power level is 5 Watts to 10 Watts, such as 5 Watts or 6 Watts.

The process 1200 is repeated at periodic time intervals (e.g. every 250 ms, every 500 ms, every second, every 1.5 seconds, every 2 seconds) throughout the treatment procedure so that the total amount of power delivered to each individual electrode 125 at the end of the treatment procedure is equal or substantially equal. The voltage source level may be based on the number of electrodes enabled (not deactivated) at any given time. In some implementations, the target power level from the voltage source in the generator 15 is 120% of the average treatment power times the number of electrodes that are enabled (not deactivated). However, other target power levels may be used as desired and/or required.

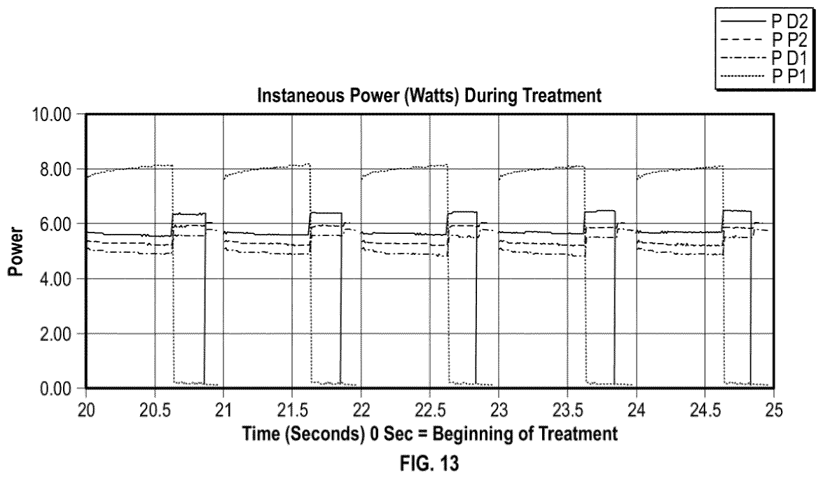

FIG. 13 illustrates an example graph of instantaneous power over time for each of four electrodes (two proximal electrodes P1, P2 and two distal electrodes D1 and D2) upon implementation of the power modulation algorithm of FIG. 12. The graph plots the instantaneous power in watts for each of the four electrodes D1, D2, P1, P2 over time. As one example, the P1 electrode may correspond to electrode 125A, the P2 electrode may correspond to electrode 125B, the D1 electrode may correspond to electrode 125C, and the D2 electrode may correspond to electrode 125D. FIG. 13 shows a snapshot over a time period that is 20 to 25 seconds from the beginning of a treatment procedure. As shown in the graph, at the start of each second of the treatment procedure, all of the electrodes are turned on (step 1205). Even though the generator 15 is set to deliver the same amount of average power (e.g., 5 W) to each electrode, because the electrodes are in contact with different tissue locations having different perivascular tissue impedance, the instantaneous power (which is determined based on voltage and current levels being applied that are sensed within the generator 15 at step 1210), that is actually delivered to each individual electrode 125 is not the same.

As shown, the power delivered to the P1 electrode is about 8 W instead of 5 W because the impedance of the perivascular tissue adjacent the P1 electrode is lower than the perivascular tissue surrounding the other electrodes. In order to not have the P1 electrode deliver more total power than the other electrodes, the control circuitry 234 adjusts the power delivery to the P1 electrode. For example, the control circuitry 234 can adjust the duty cycle of the P1 electrode (step 1215) so as to deactivate the P1 electrode earlier than the other electrodes because it has the lowest impedance. As a nonlimiting example and as shown on the graph, the P1 electrode is deactivated at about 20.6 seconds. When the P1 electrode is deactivated, the remaining electrodes P2, D1, D2 see a slight increase in the instantaneous power. At about 20.8 seconds, the control circuitry 234 deactivates the D2 electrode, as it has the next lowest impedance. To reduce clutter, the deactivation of the P2 and D1 electrodes is not shown on the graph. However, the control circuitry 234 could also adjust the duty cycles of one or both of the P2 and D1 electrodes based on the respective impedance. In the illustrated example, the process 1200 is repeated every second throughout the procedure; however, other time durations could be implemented as desired and/or required.

In some implementations, the repetition of the process 1200 is not periodic or fixed and the time durations, or duty cycles, may be adjusted throughout the procedure (e.g., at each cycle or repetition or after a certain number of cycles or repetitions). For example, the time durations, or duty cycle, for each successive interval may be determined (e.g., adjusted) based on impedance and/or temperature measurements from the previous time interval (e.g., previous 1 second interval). A similar control algorithm could be implemented for catheters using an array of ultrasound transducers instead of electrodes in order to achieve even, or equal, power distribution independent of local acoustic impedance. In some implementations, the target power level above average treatment power could be optimized to accommodate wider mismatches in impedance across the electrodes. In some implementations, different average power levels are set for individual electrodes as needed by user input or through separate sensing algorithms, such as temperature feedback or average body impedance.

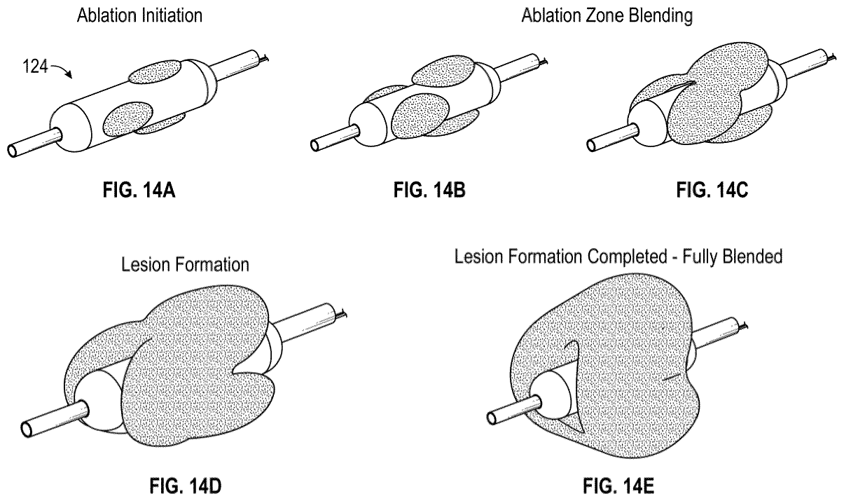

FIGS. 14A-14E show the formation of a single, continuous, blended lesion over the course of the treatment procedure based, at least in part, on implementation of the power modulation process 1200, as well as the configuration and design of the catheter 12 (e.g., geometric positioning of electrodes with both circumferential and axial separation such as a 2×2 pattern and/or continuous circulation of coolant or fluid within the catheter to cool the electrodes via jets directed at an inner surface of each of the electrodes to provide a thermodynamic benefit in which a hottest temperature is generated at a distance away from the surface of the electrodes). As shown in FIG. 14A, at the initiation of the treatment procedure, individual electrode ablation zones start to form adjacent to each individual electrode location. FIG. 14B shows the growth of the individual electrode ablation zones after a short period of time. FIGS. 14C and 14D shows that the individual electrode ablation zones are starting to blend and overlap with adjacent electrode ablation zones later on in the procedure. Finally, FIG. 14E shows the fully blended single lesion at the completion of the procedure having a desired depth, length and annularity or circumferentiality (e.g., 360-degree annularity).

In accordance with several examples, the individual electrode ablation zones are not isolated or separated and instead advantageously form a single, blended, continuous lesion. The controlled lesion geometry may advantageously facilitate uniform denervation of a perivascular space sufficient to denervate an entire organ (e.g., liver, kidney, pancreas, spleen, duodenum, jejunum or other portion of the small intestine, stomach, etc.) with one lesion formed in the perivascular space surrounding a target vessel using a catheter positioned within the target vessel.

The autonomic nervous system includes the sympathetic and parasympathetic nervous systems. The sympathetic nervous system is the component of the autonomic nervous system that is responsible for the body's “fight or flight” responses, those that can prepare the body for periods of high stress or strenuous physical exertion. One of the functions of the sympathetic nervous system, therefore, is to increase availability of glucose for rapid energy metabolism during periods of excitement or stress, and to decrease insulin secretion.

In accordance with several examples, the catheters and systems described herein are configured for modulation of nerves innervating the liver, kidneys, pancreas, spleen, small intestine, and/or other organs or tissue (e.g., organs or tissue that may affect metabolic conditions or factors). The liver can play an important role in maintaining a normal blood glucose concentration. For example, the liver can store excess glucose within its cells by forming glycogen, a large polymer of glucose. Then, if the blood glucose concentration begins to decrease too severely, glucose molecules can be separated from the stored glycogen and returned to the blood to be used as energy by other cells.

The process of breaking down glycogen into glucose is known as glycogenolysis, and is one way in which the sympathetic nervous system can increase systemic glucose. In order for glycogenolysis to occur, the enzyme phosphorylase must first be activated in order to cause phosphorylation, which allows individual glucose molecules to separate from branches of the glycogen polymer. One method of activating phosphorylase, for example, is through sympathetic stimulation of the adrenal medulla. By stimulating the sympathetic nerves that innervate the adrenal medulla, epinephrine is released. Epinephrine then promotes the formation of cyclic adenosine monophosphate (AMP), which in turn initiates a chemical reaction that activates phosphorylase. An alternative method of activating phosphorylase is through sympathetic stimulation of the pancreas. For example, phosphorylase can be activated through the release of the hormone glucagon by the alpha cells of the pancreas. Similar to epinephrine, glucagon stimulates formation of cyclic AMP, which in turn begins the chemical reaction to activate phosphorylase.

Another way in which the liver functions to maintain a normal blood glucose concentration is through the process of gluconeogenesis. When the blood glucose concentration decreases below normal, the liver will synthesize glucose from various amino acids and glycerol in order to maintain a normal blood glucose concentration. Increased sympathetic activity has been shown to increase gluconeogenesis, thereby resulting in an increased blood glucose concentration.

The parasympathetic nervous system is the second component of the autonomic nervous system and is responsible for the body's “rest and digest” functions. These “rest and digest” functions complement the “fight or flight” responses of the sympathetic nervous system. Stimulation of the parasympathetic nervous system has been associated with decreased blood glucose levels. For example, stimulation of the parasympathetic nervous system has been shown to increase insulin secretion from the beta-cells of the pancreas. Because the rate of glucose transport through cell membranes is greatly enhanced by insulin, increasing the amount of insulin secreted from the pancreas can help to lower blood glucose concentration. Similarly, parasympathetic stimulation has been shown to increase glucose uptake into the liver and thus decrease blood glucose levels.

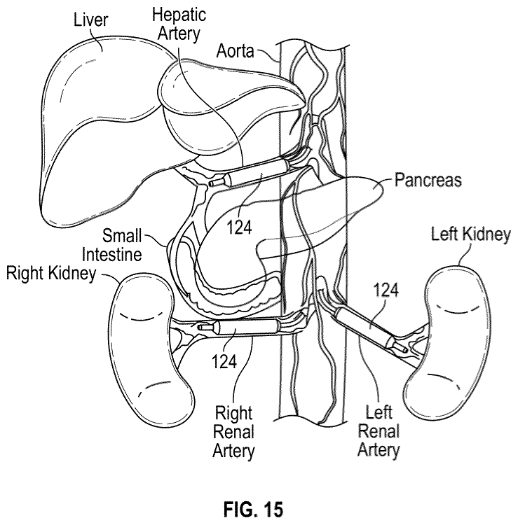

The devices described herein may be delivered to and used within various intravascular locations. The devices may be used to denervate multiple different organs (sequentially or simultaneously). FIG. 15 illustrates examples of some of the organs that may be denervated by the devices described herein (e.g., catheter 12). For example, the liver, the pancreas, the left kidney, the right kidney, and/or the small intestine (e.g., duodenum) may be denervated. FIG. 15 shows that the catheters described herein may be positioned, for example, within a right renal artery, a left renal artery, and/or a hepatic artery.

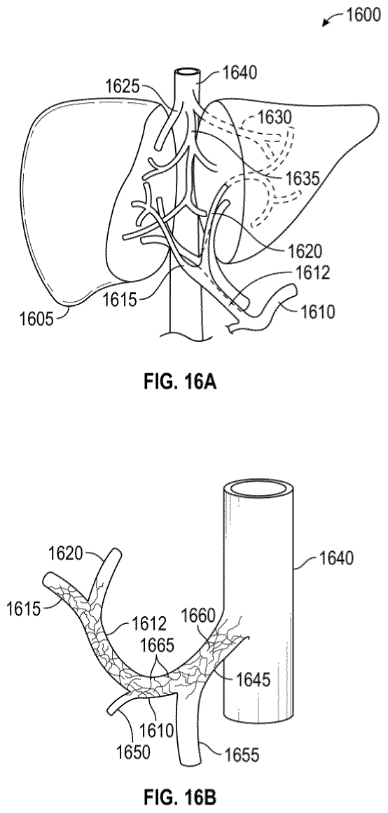

FIG. 16A illustrates a liver 1605 and vasculature 1600 of a target hepatic treatment location. The liver 1605 may be innervated along structures of or associated with the portal triad (e.g., hepatic arteries, veins, bile ducts), along which both sympathetic and parasympathetic nerve fibers may course. The vasculature includes the common hepatic artery 1610, the proper hepatic artery 1612, the right hepatic artery 1615, the left hepatic artery 1620, the right hepatic vein 1625, the left hepatic vein 1630, the middle hepatic vein 1635, and the inferior vena cava 1642. In the hepatic blood supply system, blood enters the liver by coursing through the common hepatic artery 1610, the proper hepatic artery 1612, and then either of the left hepatic artery 1620 or the right hepatic artery 1615. The right hepatic artery 1615 and the left hepatic artery 1620 (as well as the portal vein, not shown) provide blood supply to the liver 1605, and directly feed the capillary beds within the hepatic tissue of the liver 1605. The liver 1605 uses the oxygen provided by the oxygenated blood flow provided by the right hepatic artery 1615 and the left hepatic artery 1620. Deoxygenated blood from the liver 1605 leaves the liver 1605 through the right hepatic vein 1625, the left hepatic vein 1630, and the middle hepatic vein 1635, all of which empty into the inferior vena cava 1642.

FIG. 16B illustrates various arteries 1600 surrounding the liver and the various nerve systems that innervate the liver and its surrounding organs and tissue. The arteries include the abdominal aorta 1640, the celiac artery 1645, the common hepatic artery 1610, the proper hepatic artery 1612, the gastroduodenal artery 1650, the right hepatic artery 1615, the left hepatic artery 1620, and the splenic artery 1655. The various nerve systems illustrated include the celiac plexus 1660 and the hepatic plexus 1665. Blood supply to the liver is pumped from the heart into the aorta and then down through the abdominal aorta 1640 and into the celiac artery 1645. From the celiac artery 1645, the blood travels through the common hepatic artery 1610, into the proper hepatic artery 1612, then into the liver through the right hepatic artery 1615 and the left hepatic artery 1620. The common hepatic artery 1610 branches off of the celiac trunk, or artery 1645. The common hepatic artery 1610 gives rise to the gastric and gastroduodenal arteries. The nerves innervating the liver may include portions of the celiac plexus 1660 and the hepatic plexus 1665. The celiac plexus 1660 wraps around the celiac artery 1645 and continues on into the hepatic plexus 1665, which wraps around the proper hepatic artery 1612, the common hepatic artery 1610, and may continue on to the right hepatic artery 1615 and the left hepatic artery 1620. The nature of the neuroanatomy in these regions (e.g., the proximity of neural structures to the arterial lumen) is amenable to endovascular approaches for disrupting sympathetic nervous activity, including but not limited to endovascular ablation. In some anatomies, the celiac plexus 1660 and hepatic plexus 1665 adhere tightly to the walls (and some of the nerves may be embedded in the adventitia) of the arteries supplying the liver with blood, thereby rendering intra-to-extra-vascular neuromodulation particularly advantageous to modulate nerves of the celiac plexus 1660 and/or hepatic plexus 1665. In several examples, the media thickness of the vessel (e.g., hepatic artery) ranges from about 0.01 cm to about 0.25 cm. In some anatomies, at least a substantial portion of nerve fibers of the hepatic artery branches are localized within 0.5 mm to 10 mm from the lumen wall such that modulation (e.g., denervation) using an endovascular approach is effective with reduced power or energy dose requirements.

Systems and methods may be provided to identify locations along the hepatic artery that are in close proximity to adjacent structures (e.g., organs) which may influence glucose production and to modulate tissue at or near the identified locations (e.g., delivering energy using radiofrequency, ultrasound or microwave energy delivery devices sufficient to modulate nerves that innervate the liver and/or other adjacent structures that may influence glucose production (such as the pancreas, stomach, and/or small intestine (e.g., duodenum)) The modulation provided may be sufficient to reduce glucose levels (e.g., blood glucose levels), lipid levels, cholesterol levels, blood pressure levels, hepatocyte fat levels, hepatocyte fibrosis levels, etc. Modulation may be sufficient for pain relief symptoms from abdominal tumors, cancers, and growths. In various examples, portions of multiple adjacent structures (e.g., organs) may be denervated or otherwise modulated (either from a single location or from multiple locations along a portion of the hepatic artery or arteries connected or adjacent to the hepatic artery, such as the aortic artery, celiac artery, splenic artery, mesenteric arteries, renal arteries, and gastroduodenal artery).

In several examples, any of the regions (e.g., organs, arteries, nerves) identified in FIGS. 15, 16A, and 16B may be modulated according to examples described herein.

Alternatively, in one example, localized therapy is provided to the hepatic plexus, while leaving one or more of these other regions unaffected. In some examples, multiple regions (e.g., of organs, arteries, nerve systems) shown in FIGS. 15, 16A and 16B may be modulated in combination (simultaneously or sequentially), which may provide one or more synergistic effects. For example, in some examples, methods of metabolic neuromodulation treatment involve forming a single, blended lesion in the common hepatic artery as well as in one or more renal arteries, the celiac artery, the splenic artery, the gastroduodenal artery, and/or other portions or branches of the hepatic artery (e.g., proper hepatic artery, left hepatic artery, right hepatic artery) to facilitate denervation of complementary organs and structures (e.g., kidneys, pancreas, stomach, duodenum) in addition to, or as an alternative to, the liver. In some examples, if a subject has a short common hepatic artery (e.g., less than 30 mm), ablation of other vessels or portions of the hepatic artery may be desired and/or required to achieve an effective treatment. In other examples, treatment of complementary organs and structures by delivering energy in the celiac artery, splenic artery, gastroduodenal artery, one or more renal arteries, and/or other portions of the hepatic artery (e.g., proper hepatic artery, right hepatic artery, left hepatic artery) may advantageously provide one or more synergistic effects. Although several access/delivery devices are described herein that are configured for (e.g., in shape, size, flexibility, etc.) the hepatic artery, such access/delivery devices can also be used for other arteries and vessels, and in particular, other tortuous vasculature, such as the renal arteries. In addition, although devices may be described herein as neuromodulation catheters or devices and described with respect to modulation (e.g., ablation) of nerves, the catheters or other devices may be used to modulate other types of tissue (e.g., tissue lining an organ or vessel, muscle tissue, endothelial tissue, connective tissue, submucosal tissue).

Sympathetic nerves may be distributed around the hepatic arteries (or other arteries, such as the renal arteries, the celiac artery, the splenic artery, the gastroduodenal artery), and several examples of devices, systems, and methods described herein are adapted to treat these vessels. The hepatic artery passes by many adjacent structures from its origin at the celiac artery to its termination at the liver. The distance that the nerves are away from the hepatic artery or the density of nerves can be influenced by the proximity of adjacent dense structures, such as the liver, pancreas, stomach, small intestine).

In accordance with several examples, the devices and systems described herein are configured for therapeutic neuromodulation for preventing or treating disorders (such as diabetes mellitus, hypertension, obesity, factors associated with metabolic syndrome or disorders, NAFLD, NASH, and/or chronic pain) that comprise modulation of nerve fibers (e.g., nerve fibers in the perivascular space surrounding one or both renal arteries, a portion of a hepatic artery, and/or other arteries). In one example, neuromodulation decreases hepatic glucose production and/or increases hepatic glucose uptake, which in turn can result in a decrease of blood glucose levels. In one example, neuromodulation decreases hypertension. Disruption of the nerve fibers can be effected by ablating, denervating, severing, destroying, removing, desensitizing, disabling, reducing or inhibiting neural activity through, blocking, or otherwise modulating (permanently or temporarily) the nerve fibers or surrounding regions. In some examples, the disruption is carried out using one or more energy modalities. Energy modalities include, but are not limited to, microwave, bipolar or monopolar radio frequency (RF) energy, thermal energy (e.g., direct heat energy), electrical energy, extracorporeal or intracorporeal ultrasonic energy, focused ultrasound such as high-intensity focused ultrasound, laser energy, phototherapy or photodynamic therapy (e.g., in combination with one or more activation agents), cryotherapy, and chemotherapy. In some examples, the disruption of the sympathetic nerve fibers is carried out by chemicals or therapeutic agents (for example, via drug delivery), either alone or in combination with an energy modality. In some examples, the disruption is carried out by physically severing nerves with a surgical cutting, cauterizing, or chemical/drug delivery instrument in a laparoscopic or open surgical procedure, or in any combination of the above including intravascular. Several examples of the disclosure comprise disrupting cell membranes of nerve tissue. According to some examples, neuromodulation is accomplished by stimulating nerves and/or increasing neurotransmission. Stimulation, in one example, may result in nerve blocking. In other examples, stimulation enhances nerve activity (e.g., conduction of signals).

In some examples, neuromodulation of targeted nerve fibers as described herein can be used for the treatment of insulin resistance, genetic metabolic syndromes, ventricular tachycardia, atrial fibrillation or flutter, arrhythmia, inflammatory diseases, hypertension (arterial or pulmonary), obesity, hyperglycemia (including glucose tolerance), hyperlipidemia, eating disorders, NAFLD, NASH, pain, and/or endocrine diseases. In some examples, neuromodulation of targeted nerve fibers treats any combination of diabetes, insulin resistance, hypertension, obesity, and/or fatty liver. In some examples, temporary or implantable neuromodulators may be used to regulate satiety and appetite (e.g., to promote weight loss). In several examples, modulation of nervous tissue that innervates (afferently or efferently) the liver is used to treat hemochromatosis, Wilson's disease, non-alcoholic steatohepatitis (NASH), non-alcoholic fatty liver disease (NAFLD), and/or other conditions affecting the liver and/or liver metabolism. In some examples, modulation of nervous tissue that innervates (afferently or efferently) the liver (e.g., hepatic denervation) is effective for reducing whole-body sympathetic tone and resulting conditions such as hypertension, congestive heart failure, atrial fibrillation, obstructive sleep apnea, and/or renal failure, etc.

In some examples, sympathetic nerve fibers associated with the liver are selectively disrupted (e.g., ablated, denervated, disabled, severed, blocked, injured, desensitized, removed) to decrease hepatic glucose production and/or increase hepatic glucose uptake, thereby aiding in the treatment of, or reduction in the risk of, diabetes and/or related diseases or disorders. The disruption can be permanent or temporary (e.g., for a matter of several days, weeks or months). In some examples, sympathetic nerve fibers in the hepatic plexus are selectively disrupted. In some examples, sympathetic nerve fibers surrounding (e.g., within the perivascular space of) the portal triad, sympathetic nerve fibers surrounding the common hepatic artery proximal to the proper hepatic artery, sympathetic nerve fibers surrounding the proper hepatic artery, sympathetic nerve fibers in the celiac ganglion adjacent the celiac artery, other sympathetic nerve fibers that innervate or surround the liver, sympathetic nerve fibers that innervate the pancreas, sympathetic nerve fibers that innervate fat tissue (e.g., visceral fat), sympathetic nerve fibers that innervate the adrenal glands, sympathetic nerve fibers that innervate the small intestine (e.g., duodenum, jejunum, ileum), sympathetic nerve fibers that innervate the stomach (or portions thereof, such as the pylorus), sympathetic nerve fibers that innervate brown adipose tissue, sympathetic nerve fibers that innervate skeletal muscle, and/or sympathetic nerve fibers that innervate the kidneys are selectively disrupted or modulated (simultaneously or sequentially) to facilitate treatment or reduction of symptoms associated with hypertension, diabetes (e.g., diabetes mellitus), or other diseases or disorders. In some examples, the methods, devices and systems described herein are used to therapeutically modulate autonomic nerves associated with any diabetes or hypertension-relevant organs or tissues. For example, with respect to the kidneys, pancreas and duodenum, the nerves that innervate one or both structures can be neuromodulated (e.g., ablated) in addition to or instead of the nerves that innervate the liver, wherein said neuromodulation affects one or more symptoms/characteristics associated with diabetes, hypertension or other diseases or disorders. Such symptoms/characteristics include but are not limited to changes (e.g., increases or decreases) in glucose levels, cholesterol levels, lipid levels, body fat levels, fatty liver levels, triglyceride levels, hypertension levels, norepinephrine levels, insulin regulation, etc. in the blood plasma or liver or other organs. The devices and methods disclosed herein with respect to hepatic modulation (e.g., hepatic denervation) can alternatively or additionally be used for neuromodulating (e.g., denervating) at least portions of the kidneys, pancreas, duodenum, stomach or other organs and structures.

In accordance with several examples, any nerves containing autonomic fibers are modulated, including, but not limited to, the saphenous nerve, femoral nerves, lumbar nerves, median nerves, ulnar nerves, vagus nerves, and radial nerves. Nerves surrounding arteries or veins other than the hepatic artery may be additionally or alternatively be modulated such as, but not limited to, nerves surrounding the superior mesenteric artery, the inferior mesenteric artery, the femoral artery, the pelvic arteries, the portal vein, pulmonary arteries, pulmonary veins, abdominal aorta, vena cavae, splenic arteries, gastric arteries, the internal carotid artery, the internal jugular vein, the vertebral artery, renal arteries, and renal veins. Celiac arteries may also be modulated according to several examples herein.

In accordance with several examples, a therapeutic neuromodulation system (such as those described herein) is used to selectively disrupt sympathetic nerve fibers. The neuromodulation system can comprise an ablation catheter, such as the catheters described herein. An ablation catheter system may use radiofrequency (RF) energy to ablate sympathetic nerve fibers to cause neuromodulation or disruption of sympathetic communication. In other examples, an ablation catheter system uses electroporation to modulate sympathetic nerve fibers. An ablation catheter, as used herein, shall not be limited to causing ablation, but also includes devices that facilitate the modulation of nerves (e.g., partial or reversible ablation, blocking without ablation, stimulation). In some examples, a delivery catheter system delivers drugs or chemical agents to nerve fibers to modulate the nerve fibers (e.g., via chemoablation). Chemical agents used with chemoablation (or some other form of chemically-mediated neuromodulation) may, for example, include phenol, alcohol, or any other chemical agents that cause chemoablation of nerve fibers. In some examples, cryotherapy is used. For example, an ablation catheter system is provided that uses cryoablation to selectively modulate (e.g., ablate) sympathetic nerve fibers. In other examples, a delivery catheter system is used with brachytherapy to modulate the nerve fibers. The catheter systems may further utilize any combination of RF energy, ultrasonic energy, focused ultrasound (e.g., HIFU, LIFU) energy, ionizing energy (such as X-ray, proton beam, gamma rays, electron beams, and alpha rays), electroporation, drug delivery, chemoablation, cryoablation, brachytherapy, or any other modality to cause disruption or neuromodulation (e.g., ablation, denervation, stimulation) of autonomic (e.g., sympathetic or parasympathetic) nerve fibers. As discussed below, microwave energy or laser energy (or combinations of two, three or more energy sources) are used in some examples. In some examples, energy is used in conjunction with non-energy based neuromodulation (e.g., drug delivery).

In some examples, disruption or modulation of the sympathetic nerve fibers of the hepatic plexus has no effect on the parasympathetic nerve fibers surrounding the liver. In some examples, disruption or modulation of the sympathetic nerve fibers of the hepatic plexus causes a reduction of very low-density lipoprotein (VLDL) levels, thereby resulting in a beneficial effect on lipid profile. In several examples, the methods of neuromodulation comprise neuromodulation therapy to affect triglyceride, epinephrine, norepinephrine, dopamine, catecholamine, glucose, insulin, insulin-like growth factor-1 hormone, angiotensinogen, thrombopoietin, hepcidin, betatrophin, gut hormone, neuropeptide Y, pancreatic enzyme, metabolites thereof, circulating RNA, circulating DNA, high-density lipoprotein (HDL), low-density lipoprotein (LDL), and/or VLDL levels.

In accordance with some examples, neuromodulation (e.g., the disruption of sympathetic nerve fibers) is performed using a minimally invasive catheter system, such as an ablation catheter system. In some examples, an ablation catheter system for ablating nerve fibers is introduced using an intravascular (e.g., intra-arterial) approach. In one example, an ablation catheter system is used to ablate sympathetic nerve fibers surrounding (e.g., within an adventitia or perivascular area) the common hepatic artery. In some examples, the ablation catheter system is introduced via an incision in the groin to access the femoral artery. The ablation catheter system may be advanced from the femoral artery to the common hepatic artery via the iliac artery, the abdominal aorta, and the celiac artery. In other examples, any other suitable percutaneous intravascular incision point or approach is used to introduce the ablation catheter system into the arterial system (e.g., a radial approach via a radial artery or a brachial approach via a brachial artery). A laparoscopic, endoscopic, percutaneous, non-invasive or open surgical approach can also be used to effect neuromodulation treatment.

In some examples, the frequency of energy delivered is between about 50 kHz and about 20 MHz, between about 100 kHz and about 2.5 MHz, between about 400 kHz and about 1 MHz, between about 50 kHz and about 5 MHz, between about 100 kHz and about 10 MHz, between about 500 kHz and about 15 MHz, less than 50 kHz, greater than 20 MHz, between about 3 kHz and about 300 GHz, or overlapping ranges thereof. Non-RF frequencies may be used. For example, the frequency can range from about 100 Hz to about 3 kHz. In some examples, the amplitude of the voltage applied will be between about 1 volt and 1000 volts, between about 5 volts and about 500 volts, between about 10 volts and about 200 volts, between about 20 volts and about 100 volts, between about 1 volt and about 10 volts, between about 5 volts and about 20 volts, between about 10 volts and about 50 volts, between about 20 volts and about 75 volts, between about 50 volts and about 100 volts, between about 100 volts and about 500 volts, between about 200 volts and about 750 volts, between about 500 volts and about 1000 volts, less than 1 volt, greater than 1000 volts, or overlapping ranges thereof. The current density of the applied signals can have a current density between about 0.01 mA/cm² and about 100mA/cm² , between about 0.1 mA/cm² and about 50mA/cm² , between about 0.2 mA/cm² and about 10mA/cm² , between about 0.3 mA/cm² and about 5mA/cm² , less than about 0.01mA/cm² , greater than about 100mA/cm² , or overlapping ranges thereof.

If RF energy is used, then the generator 15 can generate and delivery RF signals that are pulsed or continuous. The voltage, current density, frequencies, treatment duration, and/or other treatment parameters can vary depending on whether continuous or pulsed signals are used. For example, the voltage or current amplitudes may be significantly increased for pulsed RF signals. The duty cycle for the pulsed signals can range from about 0.0001% to about 100%, from about 0.001% to about 100%, from about 0.01% to about 100%, from about 0.1% to about 100%, from about 1% to about 10%, from about 5% to about 15%, from about 10% to about 50%, from about 20% to about 60% from about 25% to about 75%, from about 50% to about 80%, from about 75% to about 100%, or overlapping ranges thereof. The pulse durations or widths of the pulsed signals can vary. For example, in some examples, the pulse durations can range from about 10 microseconds to about 1 millisecond; however, pulse durations less than 10 microseconds or greater than 1 millisecond can be used as desired and/or required. The treatment time durations can range from 1 second to 1 hour, from 5 seconds to 30 minutes, from 10 seconds to 10 minutes, from 30 seconds to 30 minutes, from 1 minute to 20 minutes, from 5 minutes to 10 minutes, from 10 minutes to 40 minutes, from 30 seconds to 90 seconds, from 5 seconds to 50 seconds, from 60 seconds to 120 seconds, less than 1 second, greater than 1 hour, or overlapping ranges thereof. The duration may vary depending on various treatment parameters (e.g., amplitude, current density, proximity, continuous or pulsed, type of nerve, size of nerve). For example, for denervation within renal arteries, the treatment duration may be 120 seconds or may range between 90 seconds and 150 seconds, whereas for denervation within hepatic arteries, the treatment duration may be 150 seconds or may range between 120 seconds and 180 seconds. In some examples, the RF energy is controlled such that delivery of the energy heats the target nerves or surrounding tissue in the range of about 60 to about 90 degrees Celsius (e.g., 60 to 75 degrees, 65 to 80 degrees, 70 to 90 degrees, or overlapping ranges thereof. In some examples, the temperature can be less than 60 or greater than 90 degrees Celsius.

In accordance with several examples disclosed herein, the catheters and systems may be configured for modulation of nerve fibers instead of or in addition to sympathetic nerve fibers in the hepatic plexus to treat diabetes or other metabolic conditions, disorders, or other diseases. For example, sympathetic nerve fibers surrounding the common hepatic artery proximal to the proper hepatic artery, sympathetic nerve fibers surrounding the celiac artery (e.g., the celiac ganglion or celiac plexus, which supplies nerve fibers to multiple organs including the pancreas, stomach, and small intestine), sympathetic nerve fibers that innervate the pancreas, sympathetic nerve fibers that innervate fat tissue (e.g., visceral fat), sympathetic nerve fibers that innervate the adrenal glands (e.g., the renal plexus or suprarenal plexus), sympathetic nerve fibers that innervate the gut, stomach or small intestine (e.g., the duodenum), sympathetic nerve fibers that innervate brown adipose tissue, sympathetic nerve fibers that innervate skeletal muscle, the gastric plexus, the splenic plexus, the splanchnic nerves, the spermatic plexus, the superior mesenteric ganglion, the lumbar ganglia, the superior or inferior mesenteric plexus, the aortic plexus, or any combination of sympathetic nerve fibers thereof may be modulated in accordance with the examples herein disclosed. In some examples, instead of being treated, these other tissues are protected from destruction during localized neuromodulation of the hepatic plexus. In some examples, one or more sympathetic nerve fibers (for example, a ganglion) can be removed (for example, pancreatic sympathectomy).

In accordance with some examples, therapeutic modulation of nerve fibers is carried out by neurostimulation of autonomic (e.g., sympathetic or parasympathetic) nerve fibers. Neurostimulation can be provided by any of the devices or systems described above (e.g., ablation catheter or delivery catheter systems) and using any approach (e.g., intravascular, laparoscopic, percutaneous, non-invasive, open surgical). In some examples, neurostimulation is provided using a temporary catheter or probe. In other examples, neurostimulation is provided using an implantable device. For example, an electrical neurostimulator can be implanted to stimulate parasympathetic nerve fibers that innervate the liver, which could advantageously result in a reduction in blood glucose levels. In some examples, the implantable neurostimulator includes an implantable pulse generator.

### VI. Other Applications and Terminology

Although the devices, systems, and methods of manufacture disclosed herein are discussed in the context of a neuromodulation catheter having an expandable outer electrode support structure or member, the expandable support structure described herein may be applied to a number of applications for minimally invasive medical procedures. A few of these applications include: dilatation balloon, magnetic resonance imaging (MRI) tracking or imaging balloon, impedance tomography balloon, optical imaging balloon, drug delivery balloon, chemical sensing balloon, expandable ultrasound transducer, thermography balloon, pulmonary vein ablation balloon, intracardiac mapping catheter, embolic protection filter, celiac ganglion block for pain management, and/or other pain management.

Terms of orientation used herein, such as “top,” “bottom,” “horizontal,” “vertical,” “longitudinal,” “lateral,” and “end” are used in the context of the illustrated example. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Various examples described herein have been presented in a range format. It should be understood that the description in range format is merely for convenience and brevity and should not be construed as an inflexible limitation on the scope of the examples. The ranges disclosed herein encompass any and all overlap, sub-ranges, and combinations thereof, as well as individual numerical values within that range. For example, description of a range such as from about 2 W to about 6 W should be considered to have specifically disclosed subranges such as from 2 to 4 W, from 3 to 5 W, from 3 to 6 W, from 4 to 6 W, etc., as well as individual numbers within that range, for example, 2, 2.5, 3, 4, 4.5, 5, 6, and any whole and partial increments therebetween. As used herein, use of “between” when describing a range includes the end values. For example, a target depth between 4 mm and 9 mm includes a target depth of 4 mm and a target depth of 9 mm. Language such as “up to,” “at least,” “greater than,” “less than,” “between,” and the like includes the number recited. Numbers preceded by a term such as “about” or “approximately” include the recited numbers (for example, “about 1” includes 1).

The terms “approximately,” “about,” and “substantially” as used herein represent an amount close to the stated amount that still performs a desired function or achieves a desired result. For example, in some examples, as the context may permit, the terms “approximately,” “about,” and “substantially” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may permit, the term “generally parallel” can refer to something that departs from exactly parallel by less than or equal to 15 degrees. As another example, in certain examples, as the context may permit, the term “generally perpendicular” can refer to something that departs from exactly perpendicular by less than or equal to 15 degrees.

### VII. Conclusion

In some examples, the systems comprise one or more of the following: means for delivering power (e.g., radiofrequency generator), means for tissue modulation (e.g., an ablation or other type of modulation catheter or delivery device), means for accessing a tissue location within a body of a subject (e.g., incision tools and/or guide catheters and/or guidewires, an ablation or other type of modulation catheter or delivery device having a sufficient length and diameter to enter through an incision (e.g., femoral or radial or carotid) and extend to a target location such as a location surrounding a renal artery, a common hepatic artery or other blood vessel described herein), etc.

Although certain examples and examples have been described herein, aspects of the methods and devices shown and described in the present disclosure may be differently combined and/or modified to form still further examples. Additionally, the methods described herein may be practiced using any device suitable for performing the recited steps. Further, the disclosure (including the figures) herein of any particular feature, aspect, method, property, characteristic, quality, attribute, element, or the like in connection with various examples can be used in all other examples set forth herein. The section headings used herein are merely provided to enhance readability and are not intended to limit the scope of the examples disclosed in a particular section to the features or elements disclosed in that section.

Furthermore, certain features that are described in this disclosure in the context of separate implementations can also be implemented in combination in a single implementation. In some examples, the systems comprise various features that are present as single features (as opposed to multiple features). For example, in one example, the system or device includes a single electrode. Conversely, various features that are described in the context of a single implementation can also be implemented in multiple implementations separately or in any suitable subcombination. Multiple features or components are provided in some examples. Moreover, although features may be described above as acting in certain combinations, one or more features from a claimed combination can, in some cases, be excised from the combination, and the combination may be claimed as a subcombination or variation of a subcombination.

Features, materials, characteristics, or groups described in conjunction with a particular aspect, example, or example are to be understood to be applicable to any other aspect, example or example described in this section or elsewhere in this specification unless incompatible therewith. All of the features disclosed in this specification (including any accompanying claims, abstract and drawings) may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination so disclosed.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Some examples have been described in connection with the accompanying drawings. The figures are drawn to scale where appropriate, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps. Any methods disclosed herein need not be performed in the order recited. The methods disclosed herein include certain actions taken by a practitioner; however, they can also include any third-party instruction of those actions, either expressly or by implication.

All of the methods and processes described above may be embodied in, and partially or fully automated via, software code modules executed by one or more general purpose computers or processors. For example, the methods described herein may be performed by one or more processors (including control circuitry) of the generator 15. The methods may be executed on the processors in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, compact disc read-only memory (CD-ROM), magnetic tape, flash drives, and optical data storage devices.

The processors may include one or more central processing units (CPUs) or processors, which may each include a conventional or proprietary microprocessor. The processors may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors (or memory units communicatively coupled thereto) may include modules comprising program instructions or algorithm steps configured for execution by the processors to perform any of all of the processes or algorithms discussed herein. The processors may be communicatively coupled to external devices (e.g., display devices, data storage devices, databases, servers, etc. over a network via a network communications interface.

In general, the word “module,” as used herein, refers to logic embodied in hardware or firmware, or to a collection of software instructions, possibly having entry and exit points, written in a programming language, such as, for example, Java, Lua, C, C #, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the computing system, for execution by the computing device. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware. Generally, any modules or programs or flowcharts described herein may refer to logical modules that may be combined with other modules or divided into sub-modules despite their physical organization or storage.

It should be understood that the examples are not to be limited to the particular forms or methods disclosed, but to the contrary, the examples are to cover all modifications, equivalents, and alternatives falling within the spirit and scope of the various examples described and the appended claims. Any methods disclosed herein need not be performed in the order recited. The methods disclosed herein include certain actions taken by a practitioner; however, they can also include any third-party instruction of those actions, either expressly or by implication. For example, actions such as “delivering a neuromodulation catheter within a hepatic artery” include “instructing the delivery of a neuromodulation catheter within a hepatic artery.”
Example 1. A method of denervating an organ by generating a continuous unitary lesion in a target perivascular space surrounding a vessel at a target intravascular treatment location using an intravascular catheter comprising a distal expandable assembly comprising a plurality of electrodes, the method comprising: positioning the distal expandable assembly of the intravascular catheter comprising the plurality of electrodes at the target intravascular treatment location; applying power to the plurality of electrodes, wherein the power applied to each of the plurality of electrodes is independently modulated based, at least in part, on at least one tissue property or characteristic of perivascular tissue surrounding each of the plurality of electrodes; and causing coolant to be continuously circulated through the distal expandable assembly so as to cool the plurality of electrodes, wherein the plurality of electrodes are geometrically positioned with both circumferential and axial separation along the distal expandable assembly such that a combination of ablation zones formed by each of the plurality of electrodes results in the continuous unitary lesion.
Example 2. The method of Example 1, wherein the continuous unitary lesion has a target depth, a target length, and a target annularity within the target perivascular space.
Example 3. The method of Example 2, wherein the target depth is between 4 mm and 9 mm beyond a wall of the vessel.
Example 4. The method of Example 2 or 3, wherein the target length is between 0.5 cm and 1.5 cm.
Example 5. The method of Example 2 or 3, wherein the target length is between 0.5 cm and 4 cm.
Example 6. The method of any of Examples 2-5, wherein the target annularity is 360-degree annularity.
Example 7. The method of any preceding example, wherein the organ is a liver.
Example 8. The method of any preceding example, wherein the organ is a kidney.
Example 9. The method of any preceding example, wherein the organ is a spleen.
Example 10. The method of any preceding example, wherein the organ is a pancreas.
Example 11. The method of any preceding example, wherein the organ is a small intestine.
Example 12. The method of any preceding example, wherein the organ is a stomach.
Example 13. The method of any preceding example, wherein the at least one tissue property or characteristic is tissue impedance.
Example 14. The method of any preceding example, wherein the plurality of electrodes comprises a 2×2 electrode pattern, wherein a first two electrodes are aligned axially but offset circumferentially, wherein a second two electrodes are aligned axially with each other but offset axially and circumferentially from the first two electrodes, and wherein the second two electrodes are offset circumferentially from each other.
Example 15. The method of any preceding example, wherein the distal expandable assembly comprises an outer expandable member and an inner expandable member, and wherein the plurality of electrodes are positioned along the outer expandable member.
Example 16. The method of Example 15, wherein causing the coolant to be continuously circulated through the distal expandable assembly causes the inner expandable member and the outer expandable member to expand and remain expanded.
Example 17. The method of Example 15 or 16, wherein the inner expandable member comprises openings positioned adjacent locations of each of the plurality of electrodes that are configured to direct jets of the coolant toward each of the plurality of electrodes so as to cool the plurality of electrodes, thereby causing a hottest temperature of the ablation zones to be at locations at a distance from a surface of each of the plurality of electrodes.
Example 18. A method of controlling lesion geometry in a target perivascular space surrounding a vessel at a target intravascular treatment location using an intravascular catheter comprising a plurality of energy delivery members so as to provide uniform denervation of the perivascular space even though perivascular tissue adjacent each of the energy delivery members may have at least one different tissue property or characteristic that affects energy delivery, the method comprising: a) applying a target average power level to each of the energy delivery members; b) sensing an actual power level being applied to each of the energy delivery members; c) adjusting a duty cycle of each of the energy delivery members based on the sensed actual power level for the respective energy delivery member; and d) repeating steps a)-c) at a periodic time interval.
Example 19. The method of Example 18, wherein applying a target average power level comprises applying a target voltage level using a single voltage source.
Example 20. The method of Example 18 or 19, wherein the target average power level is between 5 and 10 Watts.
Example 21. The method of any of Examples 18-20, wherein the target average power level is 6 Watts.
Example 22. The method of any of Examples 18-20, wherein the target average power level is 5 Watts.
Example 23. The method of any of Examples 18-22, wherein the plurality of energy delivery members comprise monopolar electrodes.
Example 24. The method of any of Examples 18-23, wherein the plurality of energy delivery members comprise ultrasound transducers.
Example 25. The method of any of Examples 18-24, wherein the periodic time interval is 1 second.
Example 26. The method of any of Examples 18-25, wherein sensing the actual power level and adjusting the duty cycle is performed in 10 millisecond (ms) increments.
Example 27. The method of any of Examples 18-26, wherein sensing the actual power level comprises sensing voltage and current levels for each of the energy delivery members.
Example 28. The method of any of Examples 18-27, wherein each of the plurality of energy delivery members comprises a sensing lead.
Example 29. The method of any of Examples 18-28, wherein the plurality of energy delivery members are spaced apart from each other axially and/or circumferentially.
Example 30. The method of any of Examples 18-29, further comprising determining a perivascular tissue impedance for each of the plurality of energy delivery members.
Example 31. The method of any of Examples 18-30, wherein the vessel is a renal artery.
Example 32. The method of any of Examples 18-30, wherein the vessel is a common hepatic artery.
Example 33. The method of any of Examples 18-32, wherein the plurality of energy delivery members comprises a plurality of electrodes that are geometrically positioned with both circumferential and axial separation along the intravascular catheter.
Example 34. The method of any of Examples 18-33, further comprising continuously cooling the plurality of energy delivery members during the method.
Example 35. The method of any of Examples 18-34, wherein the method results in lesion geometry that is blended into a continuous unitary lesion having a target depth, a target length, and a target annularity.
Example 36. The method of Example 35, wherein the target depth is between 4 mm and 9 mm.
Example 37. The method of Example 35 or 36, wherein the target length is between 0.5 cm and 2 cm.
Example 38. The method of any of Examples 35-37, wherein the target annularity is 360 degree annularity.
Example 39. A method of controlling delivery of power to a target perivascular space surrounding a vessel at a target intravascular treatment location using an intravascular catheter comprising a plurality of electrodes so as to provide uniform denervation of the perivascular space even though perivascular tissue adjacent each of the electrodes may have at least one different tissue property or characteristic that affects power delivery, the method comprising: a) applying a target average power level to each of the electrodes positioned in contact with an inner wall of the vessel; b) receiving feedback regarding an actual power level being applied to each of the electrodes; c) adjusting a duty cycle of each of the electrodes based on the feedback; and d) repeating steps a)-c).
Example 40. The method of Example 39, wherein applying a target average power level comprises applying a target voltage level using a single voltage source.
Example 41. The method of Example 39 or 40, wherein the target average power level is between 5 and 10 Watts.
Example 42. The method of any of Examples 39-41, wherein the target average power level is 6 Watts.
Example 43. The method of any of Examples 39-41, wherein the target average power level is 7 Watts.
Example 44. The method of any of Examples 39-43, wherein the plurality of electrodes are configured to function as monopolar electrodes.
Example 45. The method of any of Examples 39-44, wherein steps a)-c) are repeated at a periodic time interval.
Example 46. The method of any of Examples 39-45, wherein receiving feedback regarding the actual power level and adjusting the duty cycle is performed in 10 ms increments.
Example 47. The method of any of Examples 39-46, wherein receiving feedback regarding the actual power level comprises sensing voltage and current levels for each of the plurality of electrodes.
Example 48. The method of any of Examples 39-47, wherein each of the plurality of electrodes comprises a sensing lead.
Example 49. The method of any of Examples 39-48, wherein the plurality of electrodes are spaced apart from each other axially and/or circumferentially.
Example 50. The method of any of Examples 39-49, further comprising determining a perivascular tissue impedance for each of the plurality of electrodes.
Example 51. The method of any of Examples 39-50, wherein the vessel is a renal artery.
Example 52. The method of any of Examples 39-50, wherein the vessel is a common hepatic artery.
Example 53. A method of performing renal denervation comprising: inserting a neuromodulation catheter within a renal artery and advancing a distal expandable assembly of the neuromodulation catheter to a target treatment location within the renal artery wherein the distal expandable assembly comprises an outer expandable structure comprising a plurality of spaced-apart electrodes configured to function as monopolar electrodes, causing the distal expandable assembly to transition to an expanded configuration in which the plurality of spaced-apart electrodes are configured to contact an inner wall of the renal artery at spaced-apart locations; applying a target average power level to each of the electrodes; receiving feedback regarding an actual power level being applied to each of the electrodes; and adjusting a duty cycle of each of the electrodes based on the feedback such that each of the electrodes deliver a substantially equal amount of power over a total duration of a treatment procedure such that ablation zones formed adjacent to each of the electrodes overlap and blend to form a single blended, circumferential lesion in a perivascular space surrounding the target treatment location within the renal artery.
Example 54. The method of Example 53, wherein applying a target average power level comprises applying a target voltage level using a single voltage source.
Example 55. The method of Example 53 or 54, wherein the target average power level is between 5 and 10 Watts.
Example 56. The method of any of Examples 53-55, wherein the target average power level is 5 Watts.
Example 57. The method of any of Examples 53-56, wherein each of the plurality of electrodes comprises a sensing lead.
Example 58. The method of any of Examples 53-57, wherein the plurality of electrodes are spaced apart from each other axially and/or circumferentially.
Example 59. The method of any of Examples 53-58, wherein the total duration of the treatment procedure is between 90 seconds and 150 seconds.
Example 60. A method of performing hepatic denervation comprising: inserting a neuromodulation catheter within a hepatic artery and advancing a distal expandable assembly of the neuromodulation catheter to a target treatment location within the hepatic artery, wherein the distal expandable assembly comprises an outer expandable structure comprising a plurality of spaced-apart electrodes configured to function as monopolar electrodes, causing the distal expandable assembly to transition to an expanded configuration in which the plurality of spaced-apart electrodes are configured to contact an inner wall of the hepatic artery at spaced-apart locations; applying a target average power level to each of the electrodes; receiving feedback regarding an actual power level being applied to each of the electrodes; and adjusting a duty cycle of each of the electrodes based on the feedback such that each of the electrodes deliver a substantially equal amount of power over a total duration of a treatment procedure such that ablation zones formed at the spaced-apart locations overlap and blend to form a single blended, circumferential lesion in a perivascular space surrounding the target treatment location within the hepatic artery.
Example 61. The method of Example 60, wherein applying a target average power level comprises applying a target voltage level using a single voltage source.
Example 62. The method of Example 60 or 61, wherein the target average power level is between 5 and 10 Watts.
Example 63. The method of any of Examples 60-62, wherein the target average power level is 6 Watts.
Example 64. The method of any of Examples 60-63, wherein each of the plurality of electrodes comprises a sensing lead.
Example 65. The method of any of Examples 60-64, wherein the plurality of electrodes are spaced apart from each other axially and/or circumferentially.
Example 66. The method of any of Examples 60-65, wherein the total duration of the treatment procedure is between 120 seconds and 180 seconds.
Example 67. A neuromodulation catheter as described and/or illustrated herein.

Further disclosed herein is the subject-matter of the following clauses:
1. A neuromodulation system comprising:
   an intravascular catheter comprising:
      an elongate shaft,
      a distal expandable assembly attached to the elongate shaft, the distal expandable assembly including a plurality of energy delivery elements; and
   a generator configured to apply power to the plurality of energy delivery elements; and
   control circuitry configured to control the generator to independently modulate the power applied to each energy delivery element based on, at least in part, feedback regarding an actual power level applied to each energy delivery element, wherein to independently modulate the power applied to each energy delivery element, the control circuitry is configured to at least one of adjust a duty cycle of power applied to the respective energy delivery element or lower the power such that each of the energy delivery elements deliver a substantially equal amount of power during a treatment procedure.
   wherein the plurality of energy delivery elements are geometrically positioned along the distal expandable assembly such that a combination of ablation zones formed by each of the plurality of energy delivery elements via power applied from the generator results in a continuous unitary lesion.
2. The neuromodulation system of clause 1, wherein the feedback regarding the actual power level applied to each energy delivery element comprises at least one tissue property or characteristic.
3. The neuromodulation system of clause 2, wherein the at least one tissue property or characteristic comprises tissue impedance.
4. The neuromodulation system of any of clauses 1 through 3, wherein to receive feedback regarding the actual power level applied to each energy delivery element, the control circuitry is configured to sense at least one of a voltage or a current level for each energy delivery element.
5. The neuromodulation system of any of clauses 1 through 4, wherein the control circuitry is configured to:
   repeatedly receive the feedback regarding the actual power level over a periodic time interval; and
   automatically adjust the duty cycle over the periodic time interval.
6. The neuromodulation system of any of clauses 1 through 5, wherein the control circuitry is configured to apply a same target power level to each energy delivery element of the plurality.
7. The neuromodulation system of any of clauses 1 through 6, wherein the control circuitry is configured to:
   determine a tissue impedance sensed via one or more of the energy delivery elements of the plurality of energy delivery elements violates an impedance threshold; and
   automatically terminate an ablation cycle based on determining the tissue impedance violates the impedance threshold.
8. The neuromodulation system of any of clauses 1 through 7, wherein the control circuitry is configured to:
   determine a temperature at or adjacent to the plurality of energy delivery elements violates a temperature threshold, and
   automatically terminate an ablation cycle based on determining the temperature violates the temperature threshold.
9. The neuromodulation system of any of clauses 1 through 8, wherein the distal expandable assembly comprises an outer expandable member and an inner expandable member, and wherein the plurality of energy delivery elements are positioned along the outer expandable member.
10. The neuromodulation system of clause 9, wherein the inner expandable member and the outer expandable member are configured to transition to and remain in an expanded configuration by a fluid circulated through the distal expandable assembly.
11. The neuromodulation system of any of clauses 9 or 10, wherein the inner expandable member comprises openings positioned adjacent each energy delivery element of the plurality of energy delivery elements, the openings being configured to direct the fluid toward the plurality of energy delivery elements so as to cool the plurality of energy delivery elements, thereby causing a hottest temperature of ablation zones to be at locations at a distance from a surface of each energy delivery element of the plurality of energy delivery elements.
12. The neuromodulation system of any of clauses 1 through 11, wherein the plurality energy delivery elements comprises at least one of an electrode or an ultrasound transducer.
13. The neuromodulation system of any of clauses 1 through 12, wherein the plurality of energy delivery elements are positioned with both circumferential and axial separation along the distal expandable assembly.
14. The neuromodulation system of any of clauses 1 through 13, wherein the plurality of energy delivery elements comprises a 2×2 pattern, wherein a first two energy delivery elements are aligned axially but offset circumferentially, wherein a second two energy delivery elements are aligned axially with each other but offset axially and circumferentially from the first two energy delivery elements, and wherein the second two energy delivery elements are offset circumferentially from each other.
15. A neuromodulation system comprising:
   energy delivery circuitry; and
   control circuitry configured to:
      control the energy delivery circuitry to deliver power at a target power level to each energy delivery elements of a plurality of energy delivery elements of a catheter, and
      receive feedback regarding an actual power level applied to each energy delivery element of the plurality, and
      independently modulate the power applied to each energy delivery element of the plurality such that each of the energy delivery elements deliver a substantially equal amount of power during a treatment procedure, wherein to independently modulate the power, the control circuitry is configured to adjust a duty cycle of power applied to the respective energy delivery element.

1. A neuromodulation system comprising:
an intravascular catheter comprising:
an elongate shaft,
a distal expandable assembly attached to the elongate shaft, the distal expandable assembly including a plurality of energy delivery elements; and
a generator configured to apply power to the plurality of energy delivery elements; and
control circuitry configured to control the generator to independently modulate the power applied to each energy delivery element based on, at least in part, feedback regarding an actual power level applied to each energy delivery element, wherein to independently modulate the power applied to each energy delivery element, the control circuitry is configured to at least one of adjust a duty cycle of power applied to the respective energy delivery element or lower the power such that each of the energy delivery elements deliver a substantially equal amount of power during a treatment procedure,
wherein the plurality of energy delivery elements are geometrically positioned along the distal expandable assembly such that a combination of ablation zones formed by each of the plurality of energy delivery elements via power applied from the generator results in a continuous unitary lesion.

2. The neuromodulation system of claim 1, wherein the feedback regarding the actual power level applied to each energy delivery element comprises at least one tissue property or characteristic.

3. The neuromodulation system of claim 2, wherein the at least one tissue property or characteristic comprises tissue impedance.

4. The neuromodulation system of claim 1, wherein to receive feedback regarding the actual power level applied to each energy delivery element, the control circuitry is configured to sense at least one of a voltage or a current level for each energy delivery element.

5. The neuromodulation system of claim 1, wherein the control circuitry is configured to:
repeatedly receive the feedback regarding the actual power level over a periodic time interval; and
automatically adjust the duty cycle over the periodic time interval.

6. The neuromodulation system of claim 1, wherein the control circuitry is configured to apply a same target power level to each energy delivery element of the plurality.

7. The neuromodulation system of claim 1, wherein the control circuitry is configured to:
determine a tissue impedance sensed via one or more of the energy delivery elements of the plurality of energy delivery elements violates an impedance threshold; and
automatically terminate an ablation cycle based on determining the tissue impedance violates the impedance threshold.

8. The neuromodulation system of claim 1, wherein the control circuitry is configured to:
determine a temperature at or adjacent to the plurality of energy delivery elements violates a temperature threshold, and
automatically terminate an ablation cycle based on determining the temperature violates the temperature threshold.

9. The neuromodulation system of claim 1, wherein the distal expandable assembly comprises an outer expandable member and an inner expandable member, and wherein the plurality of energy delivery elements are positioned along the outer expandable member.

10. The neuromodulation system of claim 9, wherein the inner expandable member and the outer expandable member are configured to transition to and remain in an expanded configuration by a fluid circulated through the distal expandable assembly.

11. The neuromodulation system of claim 10, wherein the inner expandable member comprises openings positioned adjacent each energy delivery element of the plurality of energy delivery elements, the openings being configured to direct the fluid toward the plurality of energy delivery elements so as to cool the plurality of energy delivery elements, thereby causing a hottest temperature of ablation zones to be at locations at a distance from a surface of each energy delivery element of the plurality of energy delivery elements.

12. The neuromodulation system of claim 1, wherein the plurality of energy delivery elements comprises at least one of an electrode or an ultrasound transducer.

13. The neuromodulation system of claim 1, wherein the plurality of energy delivery elements are positioned with both circumferential and axial separation along the distal expandable assembly.

14. The neuromodulation system of claim 1, wherein the plurality of energy delivery elements comprises a 2×2 pattern, wherein a first two energy delivery elements are aligned axially but offset circumferentially, wherein a second two energy delivery elements are aligned axially with each other but offset axially and circumferentially from the first two energy delivery elements, and wherein the second two energy delivery elements are offset circumferentially from each other.

15. A neuromodulation system comprising:
energy delivery circuitry; and
control circuitry configured to:
control the energy delivery circuitry to deliver power at a target power level to each energy delivery element of a plurality of energy delivery elements of a catheter,
receive feedback regarding an actual power level applied to each energy delivery element of the plurality, and
independently modulate the power applied to each energy delivery element of the plurality such that each of the energy delivery elements deliver a substantially equal amount of power during a treatment procedure, wherein to independently modulate the power, the control circuitry is configured to adjust a duty cycle of power applied to the respective energy delivery element.

16. The neuromodulation system of claim 15, wherein the feedback regarding the actual power level applied to each energy delivery element comprises at least one tissue property or characteristic.

17. The neuromodulation system of claim 16, wherein the at least one tissue property or characteristic comprises tissue impedance.

18. The neuromodulation system of claim 15, wherein to receive feedback regarding the actual power level applied to each energy delivery element, the control circuitry is configured to sense at least one of a voltage or a current level for each energy delivery element.

19. The neuromodulation system of claim 15, wherein the control circuitry is configured to:
repeatedly receive the feedback regarding the actual power level over a periodic time interval; and
automatically adjust the duty cycle over the periodic time interval.

20. The neuromodulation system of claim 15, wherein the target power level of the power delivered to each energy delivery element is the same.
